SEQUENCE LISTING (4 sequences)

SEQ ID NO: 1 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

SEQ ID NO: 2 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 3 - DNA, 5629 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggcatgtcagacccgaactggcctggagagagtcctgtaccactcacgagagctgatggcactaacactgggtttccaagatatccaaatgatagtgtatatgctaactggatgctttcaccctcagcggcaaaattaatggacacgtttgatagttaacatttctttgtgaaaggtaatggactcacaaggggaagaaacatgctgagaatggaaagtctaccggccctttctttgtgaacgtcacattggccgagccgtgttcagttcccaggtggcagactcgtttttggtagtttgttttaacttccaaggtggttttacttctgatagccggtgattttccctcctagcagacatgccacaccgggtaagagctctgagtcttagtggttaagcattcctttctcttcagtgcccagcagcacccagtgttggtctgtgtccatcagtgaccaccaacattctgtgttcacatgtgtgggtccaacacttactacctggtgtatgaaattggacctgaactgttggatttttctagttgccgccaaacaaggcaaaaaaatttaaacatgaagcacacacacaaaaaaggcagtaggaaaaatgctggccctgatgacctgtccttattcagaatgagagactgcggggggggcctgggggtagtgtcaatgcccctccagggctggaggggaagaggggccccgaggatgggcctgggctcagcattcgagatcttgagaatgatttttttttaatcatgcaacctttccttaggaagacatttggttttcatcatgattaagatgattcctagatttagcacaatggagagattccatgccatctttactatgtggatggtggtatcagggaagagggctcacaagacacatttgtcccccgggcccaccacatcatcctcacgtgttcggtactgagcagccactacccctgatgagaacagtatgaagaaagggggctgttggagtcccagaattgctgacagcagaggctttgctgctgtgaatcccacctgccaccagcctgcagcacaccccacagccaagtagaggcgaaagcagtggctcatcctacctgttaggagcaggtagggcttgtactcactttaatttgaatcttatcaacttactcataaagggacaggctagctagctgtgttagaagtagcaatgacaatgaccaaggactgctacacctctgattacaattctgatgtgaaaaagatggtgtttggctcttatagagcctgtgtgaaaggcccatggatcagctcttcctgtgtttgtaatttaatgctgctacaagatgtttctgtttcttagattctgaccatgactcataagcttcttgtcattcttcattgcttgtttgtggtcacagatgcacaacactcctccagtcttgtgggggcagcttttgggaagtctcagcagctcttctggctgtgttgtcagcactgtaacttcgcagaaaagagtcggattaccaaaacactgcctgctcttcagacttaaagcactgataggacttaaaatagtctcattcaaatactgtattttatataggcatttcacaaaaacagcaaaattgtggcattttgtgaggccaaggcttggatgcgtgtgtaatagagccttgtggtgtgtgcgcacacacccagagggagagtttgaaaaatgcttattggacacgtaacctggctctaatttgggctgtttttcagatacactgtgataagttcttttacaaatatctatagacatggtaaacttttggttttcagatatgcttaatgatagtcttactaaatgcagaaataagaataaactttctcaaattattaaaaatgcctacacagtaagtgtgaattgctgcaacaggtttgttctcaggagggtaagaactccaggtctaaacagctgacccagtgatggggaatttatccttgaccaatttatccttgaccaataacctaattgtctattcctgagttataaaagtccccatccttattagctctactggaattttcatacacgtaaatgcagaagttactaagtattaagtattactgagtattaagtagtaatctgtcagttattaaaatttgtaaaatctatttatgaaaggtcattaaaccagatcatgttcctttttttgtaatcaaggtgactaagaaaatcagttgtgtaaataaaatcatgtatcataaaaaaaaaaaaaaaa

SEQ ID NO: 4 - DNA, 4174 bp, Homo sapiens
agtcccgcgaccgaagcagggcgcgcagcagcgctgagtgccccggaacgtgcgtcgcgcccccagtgtccgtcgcgtccgccgcgccccgggcggggatggggcggccagactgagcgccgcacccgccatccagacccgccggccctagccgcagtccctccagccgtggccccagcgcgcacgggcgatggcgaaggcgacgtccggtgccgcggggctgcgtctgctgttgctgctgctgctgccgctgctaggcaaagtggcattgggcctctacttctcgagggatgcttactgggagaagctgtatgtggaccaggcggccggcacgcccttgctgtacgtccatgccctgcgggacgcccctgaggaggtgcccagcttccgcctgggccagcatctctacggcacgtaccgcacacggctgcatgagaacaactggatctgcatccaggaggacaccggcctcctctaccttaaccggagcctggaccatagctcctgggagaagctcagtgtccgcaaccgcggctttcccctgctcaccgtctacctcaaggtcttcctgtcacccacatcccttcgtgagggcgagtgccagtggccaggctgtgcccgcgtatacttctccttcttcaacacctcctttccagcctgcagctccctcaagccccgggagctctgcttcccagagacaaggccctccttccgcattcgggagaaccgacccccaggcaccttccaccagttccgcctgctgcctgtgcagttcttgtgccccaacatcagcgtggcctacaggctcctggagggtgagggtctgcccttccgctgcgccccggacagcctggaggtgagcacgcgctgggccctggaccgcgagcagcgggagaagtacgagctggtggccgtgtgcaccgtgcacgccggcgcgcgcgaggaggtggtgatggtgcccttcccggtgaccgtgtacgacgaggacgactcggcgcccaccttccccgcgggcgtcgacaccgccagcgccgtggtggagttcaagcggaaggaggacaccgtggtggccacgctgcgtgtcttcgatgcagacgtggtacctgcatcaggggagctggtgaggcggtacacaagcacgctgctccccggggacacctgggcccagcagaccttccgggtggaacactggcccaacgagacctcggtccaggccaacggcagcttcgtgcgggcgaccgtacatgactataggctggttctcaaccggaacctctccatctcggagaaccgcaccatgcagctggcggtgctggtcaatgactcagacttccagggcccaggagcgggcgtcctcttgctccacttcaacgtgtcggtgctgccggtcagcctgcacctgcccagtacctactccctctccgtgagcaggagggctcgccgatttgcccagatcgggaaagtctgtgtggaaaactgccaggcattcagtggcatcaacgtccagtacaagctgcattcctctggtgccaactgcagcacgctaggggtggtcacctcagccgaggacacctcggggatcctgtttgtgaatgacaccaaggccctgcggcggcccaagtgtgccgaacttcactacatggtggtggccaccgaccagcagacctctaggcaggcccaggcccagctgcttgtaacagtggaggggtcatatgtggccgaggaggcgggctgccccctgtcctgtgcagtcagcaagagacggctggagtgtgaggagtgtggcggcctgggctccccaacaggcaggtgtgagtggaggcaaggagatggcaaagggatcaccaggaacttctccacctgctctcccagcaccaagacctgccccgacggccactgcgatgttgtggagacccaagacatcaacatttgccctcaggactgcctccggggcagcattgttgggggacacgagcctggggagccccgggggattaaagctggctatggcacctgcaactgcttccctgaggaggagaagtgcttctgcgagcccgaagacatccaggatccactgtgcgacgagctgtgccgcacggtgatcgcagccgctgtcctcttctccttcatcgtctcggtgctgctgtctgccttctgcatccactgctaccacaagtttgcccacaagccacccatctcctcagctgagatgaccttccggaggcccgcccaggccttcccggtcagctactcctcttccggtgcccgccggccctcgctggactccatggagaaccaggtctccgtggatgccttcaagatcctggaggatccaaagtgggaattccctcggaagaacttggttcttggaaaaactctaggagaaggcgaatttggaaaagtggtcaaggcaacggccttccatctgaaaggcagagcagggtacaccacggtggccgtgaagatgctgaaagagaacgcctccccgagtgagcttcgagacctgctgtcagagttcaacgtcctgaagcaggtcaaccacccacatgtcatcaaattgtatggggcctgcagccaggatggcccgctcctcctcatcgtggagtacgccaaatacggctccctgcggggcttcctccgcgagagccgcaaagtggggcctggctacctgggcagtggaggcagccgcaactccagctccctggaccacccggatgagcgggccctcaccatgggcgacctcatctcatttgcctggcagatctcacaggggatgcagtatctggccgagatgaagctcgttcatcgggacttggcagccagaaacatcctggtagctgaggggcggaagatgaagatttcggatttcggcttgtcccgagatgtttatgaagaggattcctacgtgaagaggagccagggtcggattccagttaaatggatggcaattgaatccctttttgatcatatctacaccacgcaaagtgatgtatggtcttttggtgtcctgctgtgggagatcgtgaccctagggggaaacccctatcctgggattcctcctgagcggctcttcaaccttctgaagaccggccaccggatggagaggccagacaactgcagcgaggagatgtaccgcctgatgctgcaatgctggaagcaggagccggacaaaaggccggtgtttgcggacatcagcaaagacctggagaagatgatggttaagaggagagactacttggaccttgcggcgtccactccatctgactccctgatttatgacgacggcctctcagaggaggagacaccgctggtggactgtaataatgcccccctccctcgagccctcccttccacatggattgaaaacaaactctatggtagaatttcccatgcatttactagattctagcaccgctgtcccctctgcactatccttcctctctgtgatgctttttaaaaatgtttctggtctgaacaaaaccaaagtctgctctgaacctttttatttgtaaatgtctgactttgcatccagtttacatttaggcattattgcaactatgtttttctaaaaggaagtgaaaataagtgtaattaccacattgcccagcaacttaggatggtagaggaaaaaacagatcagggcggaactctcaggggagaccaagaacaggttgaataaggcgcttctggggtgggaatcaagtcatagtacttctactttaactaagtggataaatatacaaatctggggaggtattcagttgagaaaggagccaccagcaccactcagcctgcactgggagcacagccaggttcccccagacccctcctgggcaggcaggtgcctctcagaggccacccggcactggcgagcagccactggccaagcctcagccccagtcccagccacatgtcctccatcaggggtagcgaggttgcaggagctggctggccctgggaggacgcacccccactgctgttttcacatcctttcccttacccaccttcaggacggttgtcacttatgaagtcagtgctaaagctggagcagttgctttttgaaagaacatggtctgtggtgctgtggtcttacaatggacagtaaatatggttcttgccaaaactccttcttttgtctttgattaaatactagaaatttaaaaaaaaaaaaaaa

(19) United States
(12) Patent Application Publication
(10) Pub. No.: US 2021/0290620 A1
(43) Pub. Date: Sep. 23, 2021

(54) Combinations of RET Inhibitors and mTORC1 Inhibitors and Uses Thereof for the Treatment of Cancer Mediated by Aberrant RET Activity

(71) Applicant: BLUEPRINT MEDICINES CORPORATION, Cambridge, MA (US)

(72) Inventors: Erica Evans Raab, Arlington, MA (US); Rami Rahal, Somerville, MA (US)

(21) Appl. No.: 16/613,625

(22) PCT Filed: May 15, 2018
(86) PCT No.: PCT/US2018/032794

§ 371 Date: Nov. 14, 2019

Related U.S. Application Data
(60) Provisional application No. 62/657,605, filed on Apr. 13, 2018.
(60) Provisional application No. 62/656,297, filed on Apr. 11, 2018.
(60) Provisional application No. 62/652,284, filed on Apr. 3, 2018.
(60) Provisional application No. 62/506,334, filed on May 15, 2017.

(51) Int. Cl. A61K 31/506; A61K 31/436; A61K 31/5377; A61P 35/00

(52) U.S. Cl. CPC A61K 31/506; A61P 35/00; A61K 31/5377; A61K 31/436

(57) ABSTRACT

This disclosure relates to combinations of RET inhibitors and inhibitors of mTORC1 and their use in the treatment of various cancers mediated by aberrant RET activity. Preferably, the cancer is chosen from thyroid cancer, non-small cell lung cancer, paraganglioma or intrahepatic bile duct carcinoma. The RET inhibitor preferably is BLU-667, and the mTORC1 inhibitor preferably is everolimus or AZD8055.

This application claims priority to U.S. Provisional Application No. 62/506,334, filed May 15, 2017, U.S. Provisional Application No. 62/652,284, filed Apr. 3, 2018, U.S. Provisional Application No. 62/656,297, filed Apr. 11, 2018, and U.S. Provisional Application No. 62/657,605, filed Apr. 13, 2018, the contents of which are incorporated by reference herein in its entirety.

The instant application contains a Sequence Listing which has been submitted electronically in ASCII format and is hereby incorporated by reference in its entirety. Said ASCII copy, created on May 11, 2018, is named 14320_0033-00304_SL.txt and is 32,144 bytes in size.

This disclosure relates to combinations of RET inhibitors and inhibitors of mTORC1 and their use in the treatment of various cancers.

RET is a receptor tyrosine kinase that activates multiple downstream pathways involved in cell proliferation and survival. Increasing evidence implicates aberrant activation of RET as a critical driver of tumor growth and proliferation across a broad number of solid tumors (Mulligan L M., Nat. Rev. Cancer. 14:173-186 (2014)). Oncogenic RET activation occurs via gain of function mutation or RET gene rearrangement resulting in the production of a RET fusion protein with constitutively active RET signaling that promotes ligand-independent tumor growth. Oncogenic RET activation was initially described in hereditary and sporadic thyroid cancers and subsequently in non-small cell lung cancer (NSCLC). To date, RET fusions have been implicated in several cancers, including papillary thyroid carcinoma and non-small cell lung cancer. For example, a genomics analysis on the landscape of kinase fusions identified RET fusions in breast and colon cancer patient samples, providing a therapeutic rationale for the use of RET inhibitors in multiple patient subpopulations.

Additionally, oncogenic RET rearrangements have been identified in 1-2% of NSCLC (Lipson, D. et al., Nat. Med. 18:382-384 (2012); Takeuchi, K. et al., Nat. Med. 18:378-381 (2012); Stransky, N. et al., Nat. Commun. 5:4846 (2014)). This generates a constitutively active kinase that promotes tumorigenesis. As with anaplastic lymphoma kinase (ALK) and c-ros oncogene (ROS) 1-rearranged NSCLC, RET-rearranged NSCLC typically has adenocarcinoma histology (though occasionally squamous) and occurs in young, non-smoking patients. Because diagnostic testing for RET is not standard of care, RET-rearranged patients with advanced NSCLC are treated per NCCN guidelines for epidermal growth factor receptor (EGFR-) and ALK-negative adenocarcinoma. This usually includes chemotherapy with a platinum doublet or more recently with a checkpoint inhibitor however, clinical response and overall survival specifically in RET-rearranged NSCLC with these agents is not well understood. Subsequent therapy beyond chemotherapy and checkpoint inhibitors for refractory patients per NCCN guidelines is at best supportive care or clinical trial.

Oncogenic RET activation is also associated with thyroid cancer. Thyroid cancer consists primarily of differentiated thyroid cancer (DTC; ˜90% of cases), medullary thyroid cancer (MTC; ˜5% of cases), and anaplastic thyroid cancer (<5% of cases). DTC arises sporadically from thyroid follicular cells and consists of papillary thyroid cancer (PTC) (˜80% of all thyroid cancer cases) and follicular thyroid cancer. In contrast, MTC arises from parafollicular C cells and occurs in both hereditary and sporadic forms. Oncogenic RET activation has been implicated as a driver in both MTC and PTC.

The identification of RET fusions as drivers in some cancers prompted the use of approved multi-kinase inhibitors (MKIs) with RET inhibitory activity to treat patients whose tumors express a RET fusion protein. Although encouraging response rates (˜12%-60%) (Horiike A et al., Lung Cancer 93:43-6 (March 2016); Lin J J et al., J Thorac Oncol. 11(11):2027-32 (November 2016); Gautshi O et al., J Clin Oncol. 34 (suppl; abstr 9014) (2016)) have been observed in these early studies, duration of response is typically less than a year. MKI drugs cannot always be dosed at the levels required to sufficiently inhibit RET due to toxicities that result from inhibition of targets other than RET. Further, one of the greatest challenges in treating cancer is the ability of tumor cells to become resistant to therapy. Kinase reactivation via mutation is a common mechanism of resistance. When resistance occurs, the patient's treatment options are often very limited, and the cancer progresses, unchecked, in most instances.

Recurrent gene rearrangements involving RET and a dimerization domain-encoding gene have been identified in approximately 5%-20% of sporadic papillary tumors in adults. Kinase-activating RET mutations occur in nearly all cases of hereditary MTC (87%-97%) (Machens A et al., N Engl J Med 349:1517-25 (2003); Mulligan L M et al., Nature 363(6428):458-60 (1993 Jun. 3); Mulligan L M et al., J Int Med. 238(4):343-346 (1995)) and approximately 43%-65% of sporadic MTC (Elisei R. et al., J Clin Endocrinol Metab. 93:682-687 (2008); Moura M M et al., British Journal of Cancer 100:1777-1783 (2009)). These RET mutations occur in the extracellular domain (primarily at the C634 position) which promote ligand-independent dimerization and activation of RET, and kinase domains mutations (primarily M918T, A883F or V804L/M) which promote RET auto-activation and consequent oncogenic signaling (Romei C et al., Nat Rev Endocrinol. 12(4):192-202 (2016 April)).

(1S,4R)—N—((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexanecarboxamide (Compound A) described herein is a potent and selective inhibitor of rearranged during transfection (RET) kinase and oncogenic RET mutants. In cellular systems, Compound A inhibits the kinase activity of RET oncogenic mutants with low nanomolar potency. In vivo dose-dependent antitumor efficacy with Compound A was demonstrated in several RET-driven models. Compound A is currently being investigated for use in the treatment of patients with RET-driven malignancies such as thyroid cancer, non-small cell lung cancer (NSCLC), and other advanced solid tumors.

Certain mTORC1 inhibitors have been approved in the treatment of cancer, e.g., renal cell cancer (everlolimus) and mantle cell lymphoma (temsirolimus). These inhibitors cause reduced the activity of S6 ribosomal protein kinase (S6K1) and eukaryotic initiation factor 4E-binding protein (4E-BP1), downstream effectors of mTOR, involved in protein synthesis. Inhibition of mTORC1 has been applied in estrogen-dependent and HER2+ breast cancer. Despite the efficacy of mTORC1 inhibitors as a monotherapy in certain cancers and the potential of RET inhibitors in certain cancers, there remains a need for even more effective treatment protocols in cancer. RET and mTOR are both components within the same signaling pathway, which is known to regulate various cancer-related processes including cell growth, proliferation, metabolism, and angiogenesis.

### BRIEF DESCRIPTION OF THE DRAWINGS

FIG. 1, panels A-C, depict the effect of a combination of Compound A and the mTORC1 inhibitor AZD8055 on the FTC-133 cell line. Panel A depicts the percent inhibition of cell proliferation for combinations of various amounts of Compound A and AZD8055 in combination as measured by the CTG assay. Panel B depicts the synergy score for various amounts of Compound A and AZD8055 in combination. Panel C is an isobologram depicting the effect of various amounts of Compound A and AZD8055 in combination (• line) as compared to additive results (straight line).

FIG. 2, panels A-C, depict the effect of a combination of Compound A and AZD8055 on the LC2/ad (CCDCl6-RET) cell line. Panel A depicts the percent inhibition of cell proliferation for combinations of various amounts of Compound A and AZD8055 in combination as measured by the CTG assay. Panel B depicts the synergy score for various amounts of Compound A and AZD8055 in combination. Panel C is an isobologram depicting the effect of various amounts of Compound A and AZD8055 in combination (• line) as compared to additive results (straight line).

FIG. 3, panels A-C depict the effect of a combination of Compound A and AZD8055 on a RET(C634W) cell line. Panel A depicts the percent inhibition of cell proliferation for combinations of various amounts of Compound A and AZD8055 in combination as measured by the CTG assay. Panel B depicts the synergy score for various amounts of Compound A and AZD8055 in combination. Panel C is an isobologram depicting the effect of various amounts of Compound A and AZD8055 in combination (• line) as compared to additive results (straight line).

FIG. 4, panels A-C depict the effect of a combination of Compound A and AZD8055 on the MZ-CRC-1 (RET M918T) cell line. Panel A depicts the percent inhibition of cell proliferation for combinations of various amounts of Compound A and AZD8055 in combination as measured by the CTG assay. Panel B depicts the synergy score for various amounts of Compound A and AZD8055 in combination. Panel C is an isobologram depicting the effect of various amounts of Compound A and AZD8055 in combination (• line) as compared to additive results (straight line).

FIGS. 5A, 5B, and 5C are a series of bar graphs which show the impact of Compound A on expression of DUSP6 and SPRY4 in LC2/ad (FIG. 5A), MZ-CRC-1 (FIG. 5B), and TT (FIG. 5C) cells.

FIG. 6 is a bar graph which shows the sustained decrease in expression of the MAPK target genes DUSP6 and SPRY4 in a KIF5B-RET NSCLC PDX model.

FIG. 7 is a graph which shows in vivo anti-tumor activity of Compound A in a cabozantinib-resistant tumor model generated from an engineered KIF5B-RET V804L cell line.

FIG. 8A is a graph which shows tumor size and levels of calcitonin and CEA (carcinoembryonic antigen) decrease over the course of treatment with Compound A. The patient was treated with 60 mg once daily and then received successive dose escalation up to 300 mg once daily. FIG. 8B is a CT scan at baseline (top) and after 8 weeks of Compound A treatment (bottom) demonstrating rapid reduction in tumor growth. FIG. 8C is a graph which shows tumor size and the levels of calcitonin and CEA decrease over the course of treatment with Compound A with 300 mg once daily. FIG. 8D is a CT scan of the patient's tumor at baseline (top) and after 24 weeks of Compound A treatment (bottom). FIG. 8E is a graph which shows ctDNA analysis of RET M918T levels in plasma from an MTC patient during treatment. Pre- and post-treatment tumor biopsy revealed a 93% decrease in DUSP6 and 86% decrease in SPRY4 mRNA expression after 28 days of treatment with Compound A.

FIG. 9A is a graph which shows tumor and KIF5B-RET and TP53 ctDNA reduction over the course of treatment with 200 mg once daily Compound A; FIG. 9B is a CT scan which illustrates tumor at baseline (top) and after 32 weeks of Compound A treatment (bottom).

FIG. 10A is a graph which shows the mean plasma concentration (ng/mL) vs. time (h); FIG. 10B is a bar graph which shows the percent change from baseline in mean gene expression levels of DUSP6 and SPRY4.

FIG. 11A is a bar graph which shows dose-dependent reduction in CEA in patients measured on cycle 2, day 1. FIG. 11B is a bar graph which shows dose-dependent reduction in calcitonin in patients measured on cycle 2 day 1.

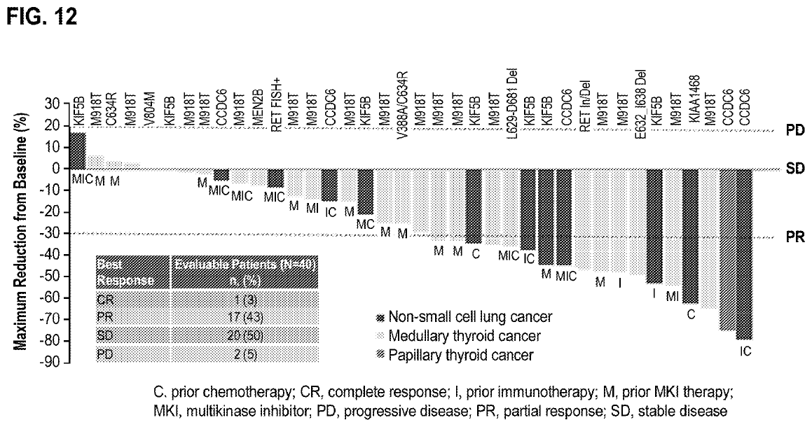

FIG. 12 is a waterfall plot which shows maximum tumor reduction—sum of diameter change from baseline percent—from patients in the phase I clinical study for Compound A.

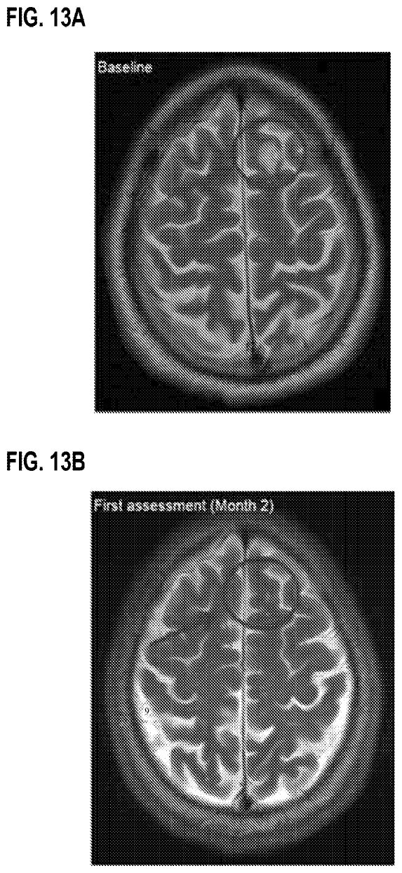

FIG. 13 is a CT scan at baseline (top) and after 8 weeks of Compound A treatment (bottom).

FIG. 14 is a chart which shows patient response rate in RET-altered NSCLC.

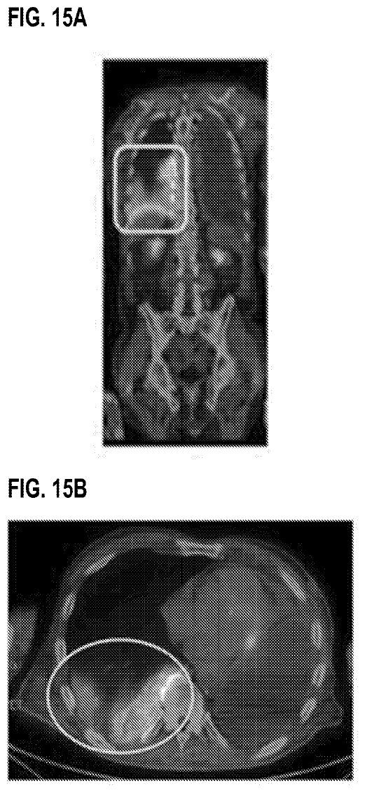

FIG. 15 is a CT scan at baseline (A-B) and after 8 weeks of Compound A treatment (bottom).

### EMBODIMENTS OF THE DISCLOSURE

### Definitions

As used herein, the terms a “patient,” “subject,” “individual,” and “host” refer to either a human or a non-human animal suffering from or suspected of suffering from a disease or disorder, e.g., cancer associated with aberrant RET activity (i.e., increased or deregulated RET activity as compared to a healthy patient, altered or additional substrate specificity as compared to wild-type RET, or increased expression of RET as compared to expression of wild-type RET).

As used herein, “treat” and “treating” such a disease or disorder refers to ameliorating at least one symptom of the disease or disorder. These terms, when used in connection with a disease such as a cancer, refer to one or more of: impeding growth of the cancer; causing the cancer to shrink by weight or volume; extending the expected survival time of the patient; inhibiting tumor growth; reducing tumor mass; reducing size or number of metastatic lesions; inhibiting the development of new metastatic lesions; prolonging survival; prolonging progression-free survival; prolonging time to progression; and/or enhancing quality of life.

As used herein, the term “preventing” when used in relation to a disease or disorder such as cancer, refers to a reduction in the frequency of, or delay in the onset of, symptoms of the disease or disorder. For example, prevention of cancer includes, e.g., reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

As used herein, the term “therapeutic effect” refers to a beneficial local or systemic effect in animals, particularly mammals, and more particularly humans, caused by administration of a compound or combination of the disclosure. The phrase “therapeutically-effective amount” means that amount of a compound or combination of the disclosure that is effective to treat a disease or disorder caused by over expression of RET or aberrant RET biological activity at a reasonable benefit/risk ratio. The therapeutically effective amount of such compound or combination will vary depending upon the subject and disease or disorder being treated, the weight and age of the subject, the severity of the disease or disorder, the manner of administration, and the like, which can readily be determined by one of skill in the art.

As used herein, an “effect marker” means DUSP6 mRNA expression, SPRY4 mRNA expression, CEA, calcitonin, KIF5B ctDNA or TP53 ctDNA.

As used herein, “developing resistance” means that when a drug is first administered to the patient, the patient's symptoms improve, whether measured by decrease in tumor volume, a decrease in the number of new lesions, or some other means that a physician uses to judge disease progression; however, those symptoms stop improving, or even worsen at some point. At that time, the patient is said to have developed resistance to the drug.

As used herein, the term “co-administering” means exposing a subject to two or more therapeutic regimens (e.g., two or more compounds). In some embodiments, two or more compounds may be administered simultaneously; in some embodiments, such compounds may be administered sequentially; in some embodiments, such compounds are administered in overlapping dosing regimens. In some embodiments, “administration” of combination therapy may involve administration of one or more compounds to a subject already receiving the other compound(s). For clarity, combination therapy does not require that individual compounds be administered together in a single composition (or even necessarily at the same time), although in some embodiments, two or more compounds may be administered together in a single combination. In some embodiments, the compounds to be co-administered are in separate dosage forms, but packaged together (e.g., in a blister pack or other pharmaceutical kit) to facilitate their co-administration.

The term “Compound A,” as used herein, means (1S,4R)—N—((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexanecarboxamide, i.e., the compound having the chemical structure:

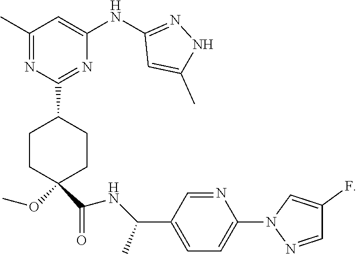 [SMILES: CO[C@]1(C(=O)N[C@@H](C)c2ccc(-n3cc(F)cn3)nc2)CC[C@H](c2nc(C)cc(Nc3cc(C)[nH]n3)n2)CC1]

In March 2017, Compound A (also known as BLU-667) entered Phase I clinical trials in the United States for the treatment of patients with thyroid cancer, non-small cell lung cancer, and other advanced solid tumors (NCT03037385). WO 2017/079140, incorporated herein by reference, describes the synthesis of Compound A (Example Compound 130) and also discloses the therapeutic activity of this molecule to inhibit, regulate, and/or modulate RET kinase (Assays, Example 10 on pp. 72-74).

In biochemical assays, Compound A inhibited the kinase activity of wild-type (WT) RET with subnanomolar potency (IC₅₀ 0.4 nM (Table 1)). Compared with the multikinase inhibitors (MKIs) cabozantinib, vandetanib, and RXDX-105 (IC₅₀ 11, 4, 3 nM, respectively), Compound A was 8- to 28-fold more potent against WT RET (Table 1). Moreover, Compound A demonstrated potent activity (IC₅₀ 0.4 nM) against common oncogenic RET alterations, including RET M918T, an activating mutation found in medullary thyroid cancer, as well as the CCDCl6-RET fusion observed in papillary carcinoma and non-small cell lung cancer (Romei C et al., Nat Rev Endocrinol 2016; 12(4):192-202, Wang R, Hu H, Pan Y, Li Y, Ye T, Li C, et al. RET fusions define a unique molecular and clinicopathologic subtype of non-small-cell lung cancer. J Clin Oncol 2012; 30(35):4352-9).

TABLE 1
Biochemical potency of Compound A and MKIs against
activated RET mutants and fusion variants

| | Biochemical IC₅₀, nM | | | | | |
|---|---|---|---|---|---|---|
| | WT | RET | RET | RET | | |
| Compound | RET | V804L | V804M | M918T | CCDC6-RET | VEGFR-2 |
| Compound A | 0.4 | 0.3 | 0.4 | 0.4 | 0.4 | 35 |
| Cabozantinib | 11 | 45 | 162 | 8 | 34 | 2 |
| Vandetanib | 4 | 3597 | 726 | 7 | 20 | 4 |
| RXDX-105 | 3 | 188 | 102 | 4 | 7 | 17 |

Abbreviations: IC₅₀, 50% inhibitory concentration; MKI, multikinase inhibitor; RET, rearranged during transfection; VEGFR-2, vascular endothelial growth factor receptor 2; WT, wild-type

The term “AZD8055,” as used herein, means the compound having the chemical structure:

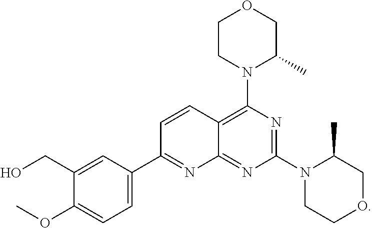 [SMILES: COc1ccc(-c2ccc3c(N4CCOC[C@@H]4C)nc(N4CCOC[C@@H]4C)nc3n2)cc1CO]

AZD8055 is an mTOR inhibitor.

The term “everolimus,” as used herein, means the compound having the chemical structure:

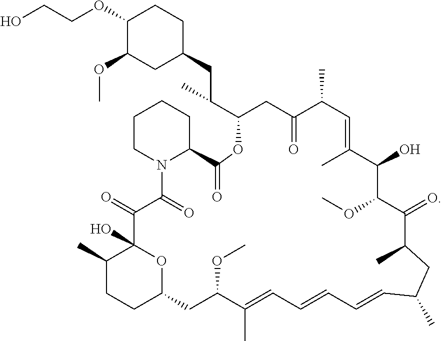 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

Everolimus is an mTOR inhibitor.

As used herein, “DOR” means duration of response.

As used herein, “PD” means progressive disease.

As used herein, “SD” means stable disease.

As used herein, “CR” means complete response.

As used herein, “ORR” means overall all response rate.

As used herein, “CBR” means clinical benefit rate.

As used herein, “PFS” means progression free survival.

As used herein, a “fusion” is a protein that results from a chromosomal translocation in which two genes are joined with an in-frame coding sequence and results in a chimeric protein. In some embodiments, a fusion is a chromosomal translocation where the kinase domain of one protein fuses to a dimerization domain of another gene.

As used herein, a “RET-altered cancer” is a cancer having an activating rearranged during transfection (RET) alteration, which drives tumorigenesis. Non-limiting examples of activating RET alterations include mutations, fusions, and copy number variations.

As used herein, a “RET fusion” is a gene rearrangement. RET rearrangements create a fusion protein juxtaposing the RET kinase domain and a dimerization domain of another protein, creating a constitutively activated dimer, which drives tumorigenesis.

As used herein, a “RET fusion protein” is the result of a gene rearrangement. RET rearrangements create a fusion protein juxtaposing the RET kinase domain and a dimerization domain of another protein, creating a constitutively activated dimer, which drives tumorigenesis.

As used herein, a “RET activating mutation” means a mutation in RET kinase which promotes ligand-independent, constitutive RET kinase activation, which drives tumorigenesis. For example, RET mutations can occur in the extracellular cysteine residues (e.g., C620R or C634R/W), which trigger aberrant receptor dimerization, or RET mutations can occur in the intracellular kinase domain.

As used herein, a “RET inhibitor” is a compound which inhibits the activity of RET kinase. RET kinase is wild-type RET kinase and/or one or more RET-altered kinases (e.g., RET fusion, RET mutation, or RET copy number variation).

Examples of RET inhibitors include, but are not limited to, Compound A, LOXO-292, cabozantinib, vandetanib, alectinib, sorafenib, levatinib, ponatinib, dovitinib, sunitinib, foretinib, sitravatinib, DS-5010, TAS0286, and RXDX-105. Additional examples of RET inhibitors include, but are not limited to, apatinib, motesanib, nintendanib, regorafenib, vatalanib, AUY-922, DCC-2157, NVP-AST487, PZ-1, RPI-1, TG101209, and SPP86.

In some embodiments, the disclosure provides a method of treating a cancer mediated by aberrant RET activity, the method comprising co-administering to a subject in need thereof a RET inhibitor and an mTORC1 inhibitor to thereby treat the cancer.

In some embodiments, aberrant RET activity is caused by a mutation in the RET gene that causes cancer. Some of these mutations can render the expressed RET protein resistant to treatment with compounds that can only inhibit wild-type RET. In some embodiments, the subject has a mutation at a position in RET that is described in Table 2.

TABLE 2
RET mutations

| Example RET Mutation | Example RET Mutation |
|---|---|
| Amino acid position 2 | Amino acid position 665 (e.g., H665Q) |
| Amino acid position 3 | Amino acid position 666 (e.g., K666E, K666M, or |
| | K666N) |
| Amino acid position 4 | Amino acid position 686 (e.g., S686N) |
| Amino acid position 5 | Amino acid position 691 (e.g., G691S) |
| Amino acid position 6 | Amino acid position 694 (e.g., R694Q) |
| Amino acid position 7 | Amino acid position 700 (e.g., M700L) |
| Amino acid position 8 | Amino acid position 706 (e.g., V706M or V706A) |
| Amino acid position 11 | Amino acid position 713 splice variant (e.g., |
| | E713K) |
| Amino acid position 12 | Amino acid position 736 (e.g., G736R) |
| Amino acid position 13 | Amino acid position 748 (e.g., G748C) |
| Amino acid position 20 | Amino acid position 750 (e.g., A750P) |
| Amino acid position 32 (e.g, S32L) | Amino acid position 765 (e.g., S765P) |
| Amino acid position 34 (e.g, D34S) | Amino acid position 766 (e.g., P766S or P766M6) |
| Amino acid position 40 (e.g., L40P) | Amino acid position 768 (e.g., E768Q or E768D) |
| Amino acid position 64 (e.g., P64L) | Amino acid position 769 (e.g., L769L) |
| Amino acid position 67 (e.g., R67H) | Amino acid position 770 (e.g, R770Q) |
| Amino acid position 114 (e.g., R114H) | Amino acid position 771 (e.g, D771N) |
| Amino acid position 136 (e.g., glutamic | Amino acid position 777 (e.g, N777S) |
| acid to stop codon) | |
| Amino acid position 145 (e.g., V145G) | Amino acid position 778 (e.g, V778T) |
| Amino acid position 180 (e.g., arginine to | Amino acid position 781 (e.g, Q781R) |
| stop codon) | |
| Amino acid position 200 | Amino acid position 790 (e.g., L790F) |
| Amino acid position 292 (e.g., V292M) | Amino acid position 791 (e.g., Y791F or Y791N) |
| Amino acid position 294 | Amino acid position 802 |
| Amino acid position 321 (e.g., G321R) | Amino acid position 804 (e.g., V804L, V804M, |
| | V804M, or V804E) |
| Amino acid position 330 (e.g., R330Q) | Amino acid position 805 (e.g., E805K) |
| Amino acid position 388 (e.g., V388A) | |
| Amino acid position 338 (e.g., T338I) | Amino acid position 806 (e.g., E806C, Y806E, |
| | Y806F, Y806S, Y806G, Y806H, Y806N, or |
| | Y806C) |
| Amino acid position 360 (e.g., R360W) | Amino acid position 818 (e.g., E818K) |
| Amino acid position 373 (e.g., alanine to | Amino acid position 819 (e.g., S819I) |
| frameshift) | |
| Amino acid position 393 (e.g., F393L) | Amino acid position 823 (e.g., G823E) |
| Amino acid position 432 | Amino acid position 826 (e.g., Y826M) |
| Δ Amino acid residues 505-506 (6-Base | Amino acid position 833 (e.g., R833C) |
| Pair In-Frame Germline Deletion in Exon | |
| 7) | |
| Amino acid position 510 (e.g, A510V) | Amino acid position 841 (e.g., P841L or P841P) |
| Amino acid position 511 (e.g., E511K) | Amino acid position 843 (e.g., E843D) |
| Amino acid position 513 (e.g., A513D) | Amino acid position 844 (e.g., R844W, R844Q, or |
| | R844L) |
| Amino acid position 515 (e.g., C515S, | Amino acid position 848 (e.g., M848T) |
| C515W) | |
| Amino acid position 525 (e.g., R525W) | Amino acid position 852 (e.g., 1852M) |
| Amino acid position 531 (e.g., C531R, or | Amino acid position 866 (e.g., A866W) |
| 9 base pair duplication) | |
| Amino acid position 532 (e.g., | Amino acid position 873 (e.g., R873W) |
| duplication) | |
| Amino acid position 533 (e.g., G533C or | Amino acid position 876 (e.g., A876V) |
| G533S) | |
| Amino acid position 550 (e.g., G550E) | Amino acid position 881 (e.g., L881V) |
| Amino acid position 591 (e.g., V591I) | Amino acid position 882 |
| Amino acid position 593 (e.g., G593E) | Amino acid position 883 (e.g., A883F, A883S, |
| | A883T, or A883T) |
| Amino acid position 600 (e.g., R600Q) | Amino acid position 884 (e.g., E884K) |
| Amino acid position 602 (e.g., I602V) | Amino acid position 886 (e.g., R886W) |
| Amino acid position 603 (e.g., K603Q or | Amino acid position 891 (e.g., S891A) |
| K603E2) | |
| Amino acid position 606 (e.g., Y606C) | Amino acid position 897 (e.g., R897Q) |
| Amino acid position 609 (e.g., C609Y, | Amino acid position 898 (e.g., D898V) |
| C609S, C609G, C609R, C609F, or | |
| C609W) | |
| Amino acid position 611 (e.g., C611R, | Amino acid position 901 (e.g., E901K) |
| C611S, C611G, C611Y, C611F, or | |
| C611W) | |
| Amino acid position 618 (e.g, C618S, | Amino acid position 904 (e.g., S904F or S904C2) |
| C618Y, C618R, C618Y, C618G, C618F, | |
| C618W) | |
| Amino acid position 619 (e.g., F619F) | Amino acid position 907 (e.g., K907E or K907M) |
| Amino acid position 620 (e.g, C620S, | Amino acid position 908 (e.g., R908K) |
| C620W, C620R, C620G, C620L, C620Y, | |
| C620F) | |
| Amino acid position 623 (e.g., E623K) | Amino acid position 911 (e.g, G911D) |
| Amino acid position 624 (e.g., D624N) | Amino acid position 912 (e.g, R912P, R912Q) |
| Amino acid position 629 (e.g., L629P) | |
| Amino acid position 630 (e.g., C630A, | Amino acid position 918 (e.g., M918T, M918V, or |
| C630R, C630S, C630Y, or C630F) | M918L6) |
| Amino acid position 631 (e.g., D631N, | Amino acid position 919 (e.g., A919V) |
| D631Y, D631A, D631G, D631V, D631E, | |
| or D631_R635DELINSG) | |
| Amino acid position 632 (e.g., E632K or | Amino acid position 921 (e.g., E921K) |
| E632G5) | |
| Δ Amino acid residues 632-633 (6-Base | Amino acid position 922 (e.g., S922P or S922Y) |
| Pair In-Frame Germline Deletion in Exon | |
| 11) | |
| Amino acid position 633 (e.g., 9 base pair | Amino acid position 930 (e.g., T930M) |
| duplication) | |
| Amino acid position 634 (e.g, C634W, | Amino acid position 961 (e.g, F961L) |
| C634Y, C634S, C634R, C634F, C634G, | |
| C634L, C634A, or C634T, or an insertion | |
| ELCR2, or a 12 base pair duplication) | |
| Amino acid position 635 (e.g., R635G) | Amino acid position 972 (e.g., R972G) |
| Amino acid position 636 (e.g., T636P or | Amino acid position 982 (e.g, R982C) |
| T636M4) | |
| Amino acid position 637 (e.g., V637R) | |
| Amino acid position 640 (e.g., A640G) | Amino acid position 1009 (e.g., M1009V) |
| Amino acid position 641 (e.g, A641S or | Amino acid position 1017 (e.g., Dl017N) |
| A641T8) | |
| Amino acid position 648 (e.g., V6481) | Amino acid position 1041 (e.g., V1041G) |
| Amino acid position 649 (e.g, S649L) | Amino acid position 1064 (e.g., M1064T) |
| Amino acid position 664 (e.g., A664D) | RET + 3 |

Some of the RET mutations in Table 2 are discussed in: U.S. Patent Application Publication No. 2014/0272951; Krampitz et al., Cancer 120:1920-31 (2014); Latteyer et al., J Clin. Endocrinol. Metab. 101(3): 1016-22 (2016); Silva et al. Endocrine 49.2:366-72 (2015); Jovanovic et al., Prilozi 36(1):93-107 (2015); Qi et al., Oncotarget 6(32):33993-4003 (2015); Kim et al. ACTA ENDOCRINOLOGICA-BUCHAREST 11.2, 189-194, (2015); Cecchirini et al. Oncogene, 14:2609-12 (1997); Karrasch et al., Eur. Thyroid J 5(1):73-77 (2016); Scollo et al., Endocr. J 63:87-91 (2016); and Wells et al., Thyroid 25:567-610 (2015).

R525W and A513D may act in combination with S891A to enhance oncogenic activity.

In some embodiments, the RET mutation has point mutations at the 804 gatekeeper residue in the RET protein and/or at residues at or near the gatekeeper residue. For example, in some embodiments, these resistant mutations that encode an aberrant RET may be at one or more of the 804, 806, 810, 865, 870, 891, and 918 amino acid residues. In some embodiments, such resistant mutations may include one or more of the following amino acid mutations: V804L; V804M; V804E; Y806C; Y806S; Y806H; Y806N; G810R; G810S; L865V; L870F; S891A; and M918T. For example, in some embodiments, the aberrant RET activity is caused by a M918T mutation.

Additionally, mutations at cysteine amino acids 609, 611, 618, 620, 630 and/or 634 of RET have been identified in patients suffering from medullary thyroid cancer and are believed to be oncogenic. In some embodiments, the RET cysteine variants (affecting C609, C611, C618, and C620) are known as “Janus mutations.” For example, in some embodiments, RET with one or more of the following additional mutations is associated with aberrant RET activity: C618R; C618S; C634Y; C634W; and C634R. In some embodiments, the aberrant RET activity is caused by a C634W mutation.

In some embodiments, aberrant RET activity is caused by over-expression of the wild-type RET protein. This may be due to increased RET gene copy number, a defect in regulatory cell machinery that controls RET mRNA or RET protein level, or a mutation in the promoter region of the RET gene that causes increased RET expression.

In some embodiments, aberrant RET activity is caused by oncogenic RET fusions. Such fusions may be with either wild-type RET or any mutant form of RET disclosed above. In some embodiments, the subject has a fusion between RET and a RET fusion partner listed in Table 3, e.g., comprises a fusion protein that comprises RET or a fragment thereof and a protein of Table 3 or fragment thereof. In some embodiments, the fusion partner is N-terminal or C-terminal of RET. In some embodiments, a subset of the subject's cells, e.g., a subset of the subject's tumor cells, comprise the RET alteration. In some embodiments, a subset of the subject's cells, e.g., a subset of the subject's tumor cells, are RET-altered. In some embodiments, the subject has a cancer listed in Table 3, e.g., the subject has both a RET mutation and a cancer listed in Table 3.

TABLE 3
RET Fusions

| RET fusion partner | Exemplary cancers in which the fusion is found |
|---|---|
| BCR | Chronic Myelomonocytic Leukemia (CMML) |
| CLIP 1 | Adenocarcinoma |
| KIF5B | NSCLC, Ovarian Cancer, Spitzoid Neoplasm; Lung |
| | Adenocarcinoma, Adenosquamous Carcinomas |
| CCDC6 | NSCLC, Colon Cancer, Papillary Thyroid Cancer; Adenocarcinoma; |
| | Lung Adenocarcinoma; Metastatic Colorectal Cancer; |
| | Adenosquamous Carcinoma, Metastatic papillary thyroid cancer |
| PTClex9 | Metastatic papillary thyroid cancer |
| NCOA4 | Papillary Thyroid Cancer, NSCLC, Colon Cancer, |
| | Salivary Gland Cancer, Metastatic Colorectal Cancer; Lung |
| | Adenocarcinoma, Adenosquamous Carcinomas; Diffuse Sclerosing |
| | Variant of Papillary Thyroid Cancer |
| TRIM33 | NSCLC, Papillary Thyroid Cancer |
| ERC1 | Papillary Thyroid Cancer, Breast Cancer |
| FGFRIOP | CMML, Primary Myelofibrosis with secondary Acute Myeloid |
| | Leukemia |
| MBD1 | Papillary Thyroid Cancer |
| RAB61P2 | Papillary Thyroid Cancer |
| PRKAR1A | Papillary Thyroid Cancer |
| TRIM24 | Papillary Thyroid Cancer |
| KTN1 | Papillary Thyroid Cancer |
| GOLGA5 | Papillary Thyroid Cancer, Spitzoid Neoplasms |
| HOOK3 | Papillary Thyroid Cancer |
| KIAA1468 | Papillary Thyroid Cancer, Lung Adenocarcinoma |
| TRIM27 | Papillary Thyroid Cancer |
| AKAP13 | Papillary Thyroid Cancer |
| FKBP15 | Papillary Thyroid Cancer |
| SPECC1L | Papillary Thyroid Cancer, Thyroid Gland Carcinoma |
| TBL1XR1 | Papillary Thyroid Cancer, Thyroid Gland Carcinoma |
| CEP55 | Diffuse Gastric Cancer |
| CUX1 | Lung Adenocarcinoma |
| ACBD5 | Papillary Thyroid Carcinoma |
| MYH13 | Medullary Thyroid Carcinoma |
| PIBF1 | Bronchiolus Lung Cell Carcinoma |
| KIAA1217 | Papillary Thyroid Cancer, Lung Adenocarcinoma, NSCLC |
| MPRIP | NSCLC |

Some of the RET fusions in Table 3 are discussed in: Grubbs et al., J Clin Endocrinol Metab., 100:788-93 (2015); Halkova et al., Human Pathology 46:1962-69 (2015); U.S. Pat. Nos. 9,297,011; 9,216,172; Le Rolle et al., Oncotarget 6(30):28929-37 (2015); Antonescu et al., Am J Surg Pathol 39(7):957-67 (2015); U.S. Patent Application Publication No. 2015/0177246; U.S. Patent Application Publication No. 2015/0057335; Japanese Patent Application Publication No. 2015/109806A; Chinese Patent Application Publication No. 105255927A; Fang, et al., Journal of Thoracic Oncology 11.2 (2016): S21-S22; European Patent Application Publication No. EP3037547A1; Lee et al., Oncotarget DOI: 10.18632/oncotarget.9137, e-published ahead of printing, 2016; Saito et al., Cancer Science 107:713-20 (2016); Pirker et al., Transl Lung Cancer Res, 4(6):797-800 (2015); and Joung et al., Histopathology 69(1):45-53 (2016).

A person of ordinary skill in the art may determine if a subject possesses a RET-altered cell, cancer, gene, or gene product, e.g., having a mutation, e.g., a fusion, deletion, insertion, translocation, frameshift, duplication, point mutation, and/or rearrangement, e.g., using a method chosen from hybridization-based methods, amplification-based methods, microarray analysis, flow cytometry analysis, DNA sequencing, next-generation sequencing (NGS), primer extension, PCR, in situ hybridization, fluorescent in situ hybridization, dot blot, and Southern blot.

To detect a fusion, primary tumor samples may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumors samples.

Next, genes in which 5′ exons are expressed at significantly different levels than 3′ exons are identified. A sliding frame is used to identify the breakpoint within an individual sample. Specifically, at each iteration, an incremental breakpoint divides the gene into 5′ and 3′ regions, and a t-statistic is used to measure the difference in expression (if any) between the two regions. The breakpoint with the maximal t-statistic is chosen as the likely fusion breakpoint. As used herein, “breakpoint” is the boundary at which two different genes are fused. It is sometimes referred to as a “fusion point.” The location where the difference in exon expression is maximal between 5′ and 3′ is the inferred breakpoint of the fusion. Thousands of tumor samples can be rapidly profiled in this manner, generating a list of fusion candidates (ranked by t-statistic). High-ranking candidates can then be validated and fusion partners identified by examining the raw RNA-seq data sets, and identifying chimeric pairs and/or split reads which support the fusion. Candidate fusions can then be experimentally confirmed as described below.

Alternatively, the methods described in Wang L et al., Genes Chromosomes Cancer 51(2):127-39 (2012). doi: 10.1002/gcc.20937, Epub 2011 Oct. 27; and Suehara Y et al., Clin Cancer Res. 18(24):6599-608 (2012). doi: 10.1158/1078-0432.CCR-12-0838, Epub 2012 Oct. 10 can also be used.

It has been proposed that the inclusion of a pharmacodynamic assessment of molecularly targeted therapies in clinical trials can streamline the drug development process (Tan D S et al., Cancer J 15(5):406-20 (2009); Sarker D & Workman P. Adv Cancer Res 96:213-68 (2007)). Pharmacodynamic biomarkers have been successfully utilized for the clinical development of kinase inhibitors, including imatinib and gefitinib (Sarker D & Workman P. Adv Cancer Res 96:213-68 (2007); Baselga J et al., J Clin Oncol 23(23):5323-33 (2005); Druker B J et al., N Engl J Med 344(14):1031-7 (2001)). As described herein, Compound A dose-dependently inhibited RET and SHC activation, which mirrored the inhibition of DUSP6 and SPRY4 transcription across RET-driven preclinical models, indicating that these transcripts can serve as biomarkers for RET inhibitory activity. The translational capability of these markers was established in this study in which MTC tumor shrinkage induced by Compound A treatment was associated with efficient inhibition of DUSP6 and SPRY4 expression within the tumor tissue.

RET has two primary protein and mRNA isoforms, named RET51 and RET9. In some embodiments, RET has a sequence of isoform RET51 (SEQ ID NO: 1). The kinase domain corresponds to amino acids 724-1016 of SEQ ID NO: 1. Unless otherwise indicated, the amino acid positions described herein refer to the numbering of RET51 in SEQ ID NO: 1.

In some embodiments, RET has a sequence of isoform RET9 (SEQ ID NO: 2), wherein the kinase domain is 724-1016.

In some embodiments, RET51 is encoded by a nucleic acid having the sequence of SEQ ID NO: 3.

In some embodiments, RET9 is encoded by a nucleic acid having the sequence of SEQ ID NO: 4.

The RET inhibitor utilized in some embodiments may be a RET-selective inhibitor that only inhibits wild-type RET, an inhibitor that only inhibits one or more mutant forms of RET, or an inhibitor that only inhibits wild-type RET and one or more mutant forms of RET and has little inhibitory activity against other kinases. The RET inhibitor utilized in some embodiments may also be an inhibitor that inhibits wild-type RET and other kinases or an inhibitor that inhibits both wild-type and one or more mutant forms of RET and has inhibitory activity against other kinases.

RET inhibitors that may be utilized in some embodiments include those that are well-known in the art, e.g., vandetanib, cabozantinib, sunitinib, sorafenib, ponatinib, RXDX-105, apatinib, lenvatinib, Compound A and compounds disclosed in US 2017/0121312 or US 2013/0096136, alectinib, dovitinib, regorafenib, and nintedanib, as well as compounds disclosed in PCT publications WO2017/079140, WO 2017/011776, WO 2016/127074, WO 2016/075224, WO 2016/038552, WO 2016/037578, WO 2015/079251, WO 2015/006875, 2015/006875, WO 2014/141187, WO 2014/050781, WO 2009/100536, and compounds disclosed in any of WO20176/161269, WO2018/017983, and WO2018/022761.

In some embodiments, the RET-selective inhibitor is Compound A. Compound A is a RET-selective inhibitor that only inhibits wild-type RET and one or more mutant forms of RET and has little inhibitory activity against other kinases

As used herein, a “mTORC1 inhibitor” is a compound that inhibits the activity of mammalian target of rapamycin complex 1 (mTORC1).

The mTORC1 inhibitor utilized in some embodiments, may be chosen from any known mTORC1 inhibitor. Such inhibitors may have activity against mTORC2 or not. Approved mTORC1 inhibitors that may be utilized in some embodiments of the disclosure include sirolimus (solid tumor), temsirolimus/CC1-779 (advanced renal cell carcinoma), and everolimus (advanced renal cell carcinoma). Other inhibitors that may be utilized in some embodiments include dactolisib/BEZ235 (breast cancer, solid tumor), omipalisib/GSK2126458 (solid tumor), XL765/SAR245409 (glioma), AZD8055 (glioma), INK128/MLN0128 (endometrial neoplasms), OSI027 (solid tumor or lymphoma), and various RapaLinks (Rodrik-Outmezguine V S, Okaniwa M, Yao Z, et al., Nature. 2016; 534(7606):272-6) (gliomoblastoma). In some embodiments, the mTORC1 inhibitor is rabamycin or a formulation of rapamycin such as Nab™-rapamycin or analog, e.g., ridaforolimus.

In some embodiments, the mTORC1 inhibitor is an allosteric inhibitor.

In some embodiments, the mTORC1 inhibitor is everolimus or AZD8055. In some embodiments, the mTORC1 inhibitor is everolimus. In some embodiments, the mTORC1 inhibitor is AZD8055.

In some embodiments, the RET inhibitor is Compound A and the mTORC1 inhibitor is everolimus or AZD8055. In some embodiments, the RET inhibitor is Compound A and the mTORC1 inhibitor is everolimus. In some embodiments, the RET inhibitor is Compound A and the mTORC1 inhibitor is AZD8055.

In some embodiments, each of the mTORC1 and RET inhibitors is administered once a day.

In some embodiments, the mTORC1 inhibitor is administered twice per day and the RET inhibitor is administered once a day.

In some embodiments, the mTORC1 inhibitor is administered once per day and the RET inhibitor is administered twice a day.

In some embodiments, each of the mTORC1 and RET inhibitors is administered twice a day.

In some embodiments, the cancer to be treated is any cancer that is characterized by aberrant RET activity. Many cancers have been linked to aberrant RET expression (Kato et al., Clin. Cancer Res. 23(8):1988-97 (2017)). Non-limiting examples of “cancer,” as used herein, include lung cancer, head and neck cancer, gastrointestinal cancer, breast cancer, skin cancer, genitourinary tract cancer, gynecological cancer, hematological cancer, central nervous system (CNS) cancer, peripheral nervous system cancer, endometrial cancer, colorectal cancer, bone cancer, sarcoma, spitzoid neoplasm, adenosquamous carcinoma, pheochromocytoma (PCC), hepatocellular carcinoma, multiple endocrine neoplasia (MEN2A and MEN2B), and inflammatory myofibroblastic tumor. For other examples of cancers linked to aberrant RET expression, see Nature Reviews Cancer 14:173-86 (2014).

Additional non-limiting examples of cancer include hemangiopericytoma, differentiated thyroid carcinoma, anaplastic thyroid carcinoma, lung carcinosarcoma, ureter urothelial carcinoma, uterine carcinosarcoma, basal cell carcinoma, Merkel cell carcinoma, atypical lung carcinoma, fallopian tube adenocarcinoma, ovarian epithelial carcinoma, salivary gland adenocarcinoma, meningioma, duodenal adenocarcinoma, cervical adenocarcinoma, adrenal carcinoma, gastroesophageal junction carcinoma, cutaneous squamous cell carcinoma, pancreatic ductal adenocarcinoma, prostate adenocarcinoma, esophageal adenocarcinoma, endometrial adenocarcinoma, ovarian serous carcinoma, carcinoma unknown primary, bladder urothelial (transition cell) carcinoma, lung squamous cell carcinoma, colorectal adenocarcinoma, head and neck squamous cell carcinoma, and gastric adenocarcinoma.

In some embodiments, the cancer to be treated is a cancer described in Table 3.

In some embodiments, the condition associated with aberrant RET activity is a thyroid cancer (e.g., papillary thyroid carcinoma, thyroid adenocarcinoma, or MTC, e.g., familial MTC), lung cancer (e.g., lung adenocarcinoma, small-cell lung carcinoma, or non-small cell lung carcinoma), breast cancer (e.g., estrogen receptor-positive tumors and endocrine-resistant tumors e.g., resistant to oestrogen modulators such as tamoxifen, agents that block oestrogen biosynthesis such as aromatase inhibitors, and oestrogen receptor antagonists such as fulvestrant), pancreatic cancer (e.g., carcinoma of the pancreas or pancreatic ductal carcinoma), hematopoietic cancer, e.g., a leukemia (e.g., chronic myelomonocytic leukemia or acute myeloid leukemia), colon cancer (e.g., colon carcinoma), melanoma (e.g., cutaneous or desmoplastic malignant melanomas), prostate cancer, renal cancer (e.g., renal cell carcinoma), and head and neck tumors, neuroblastoma, ganglioneuroma (e.g., ganglioneuroma of the mouth or gut), colon cancer (e.g., sporadic colon cancers), MEN2A (multiple endocrine neoplasia type 2A), or MEN2B (multiple endocrine neoplasia type 2B).

In some embodiments, the cancer to be treated is chosen from papillary thyroid carcinoma (PTC), medullary thyroid cancer (MTC), and non-small cell lung cancer.

In some embodiments, MEN2A is associated with pheochromocytoma and parathyroid hyperplasia. Substitutions of cysteines in RET are found in subjects with MEN2A and also frequently in FMTC. RET extracellular domain exon 8 mutations, such as G533C) or the RET intracellular domain (residues E768, L790, Y791, V804, and S891) are associated with FMTC or MEN2A. Substitutions in the RET kinase domain, Met918 to Thr (M918T) or A883F are found in subjects with MEN2B. RET M918T and RET A883F are also found in sporadic MTC.

In some embodiments, the MEN2A is characterized by MTC and includes adrenal tumor pheochromocytoma.

In some embodiments, MEN2B is associated with mucosal neuromas, pheochromocytomas, intestinal ganglioneuromas and marfanoid habitus.

In some embodiments, the lung cancer is chosen from small cell lung cancer (SCLC), lung adenocarcinoma, non-small cell lung cancer (NSCLC), bronchioles lung cell carcinoma, and mesothelioma.

In some embodiments, the head and neck cancer is chosen from thyroid cancer and cancer of the salivary gland. In some embodiments, the thyroid cancer is chosen from papillary thyroid carcinoma (PTC), metastatic papillary thyroid cancer, medullary thyroid cancer (MTC), diffuse sclerosing variant of papillary thyroid cancer, and thyroid gland carcinoma. In some embodiments, the cancer is familial medullary thyroid cancer.

In some embodiments, the gastrointestinal cancer is chosen from esophageal cancer, esophagogastric cancer, gastrointestinal stromal tumor (e.g., imatinib-resistant gastrointestinal stromal tumor), small bowel cancer, diffuse gastric cancer, and ampullary carcinoma.

In some embodiments, the breast cancer is metastatic breast cancer. In some embodiments, skin cancer is melanoma or non-melanoma.

In some embodiments, the genitourinary tract cancer is chosen from colon cancer, metastatic colon cancer, bladder cancer, renal cell carcinoma (RCC), prostate cancer, hepatobiliary cancer, intrahepatic bile duct cancer, adrenocortical carcinoma, pancreatic cancer, and pancreatic ductal adenocarcinoma.

In some embodiments, the gynecological cancer is chosen from uterine sarcoma, germ cell tumor, cervical cancer, rectal cancer, testicular cancer, and ovarian cancer.

In some embodiments, the hematological cancer is chosen from leukemia, primary myelofibrosis with secondary acute myeloid leukemia, myelodysplasia (MDS), non-Hodgkin lymphoma, chronic myeloid leukemia, Philadelphia chromosome-positive acute lymphoblastic leukemia, and chronic myelomonocytic leukemia (CMML).

In some embodiments, the peripheral nervous system cancer is paraganglioma. In some embodiments, the endometrial cancer isendometrial adenocarcinoma. In some embodiments, the sarcoma is a soft tissue sarcoma. In some embodiments, the central nervous system (CNS) cancer is chosen from brain cancer associated with lung cancer and glioma. In some embodiments, the cancer is a glioma. In some embodiments, the cancer is chosen from glioblastoma multiforme, anaplastic astrocytoma, anaplastic oligodendroglioma, malignant glioma, and brainstem glioma. In some embodiments, the cancer is chosen from mixed gliomas, malignant gliomas, and glioblastoma multiforme.

In some embodiments, the cancer is chosen from breast cancer, advanced solid tumor, and Cowden Syndrome.

In some embodiments, the cancer is endometrial neoplasms.

In some embodiments, the cancer is solid tumor or lymphoma

In some embodiments, the cancer is solid tumor. In some embodiments, the solid tumor is chosen from sarcoma, pancreas (adenocarcinoma), colorectal cancer, hepatocellular cancer, neuroendocrine cancer, and non-small cell lung cancer.

In some embodiments, the cancer is chosen from renal cell carcinoma, gastric cancer, non-small cell lung cancer, and hepatocellular carcinoma.

In some embodiments, the cancer is mantle cell lymphoma.

In some embodiments, the cancer is soft-tissue or bone sarcoma.

Lung cancer is known to spread to the brain in about 40 percent of cases in which a metastasis has occurred. With lung cancer, this is considered stage 4 of the disease, and the average survival time with brain metastases is usually less than a year. Lung cancers with metastases to the brain have a relatively poor prognosis, e.g., chemotherapy drugs. Brain metastases are difficult to treat for many reasons. Often, by the time the patient first exhibits symptoms, they already have multiple lesions. Brain metastases tend to be very aggressive. The brain has many defenses to reduce the penetration of external substances. Specifically, the blood-brain-barrier prevents many medications from entering the brain. Treatment options for brain metastases may damage surrounding normal tissue and have significant impact on the quality of life.

In some embodiments, the cancer is brain metastasis associated with lung cancer.

In some embodiments, the cancer is a “RET-altered cancer,” which, as used herein, means the cancer has an activating RET alteration. In some embodiments, the RET-altered cancer has a RET mutation or a RET gene rearrangement. In some embodiments, the RET-altered cancer is a RET-altered solid tumor.

Methods for determining aberrant RET activity in a patient are known in that art and any such methods may be utilized to make such determination including: detection of a RET gene fusion, an mRNA transcribed from such gene fusion, or the fusion protein encoded by such mRNA; detection of a mutant RET gene, an mRNA transcribed from such gene, or the protein encoded by such mRNA; in vitro measurement of RET kinase activity associated with the cancer compared to a normal control; detection of wild-type RET mRNA or protein levels, etc.

Some example embodiments of the disclosure include the following:

1. A method of treating a cancer mediated by aberrant RET activity in a subject in need thereof, the method comprising co-administering to the subject a RET inhibitor and an mTORC1 inhibitor.
2. The method of embodiment 1, wherein the aberrant RET activity is attributed to an oncogenic mutant of RET or an oncogenic RET fusion.
3. The method of embodiment 1 or 2, wherein the aberrant RET activity is attributed to:

a RET mutation chosen from C634W, V804L, V804M, V804E, Y806C, Y806S, Y806H, Y806N, G810R, G810S, L865V, L870F, S891A and M918T; or

a RET fusion chosen from KIF5B-RET and CCDCl6-RET.

4. The method of any one of embodiments 1 to 3, wherein the aberrant RET activity is attributed to:

a RET mutation chosen from C634W and M918T; or

a CCDCl6-RET fusion.

5. The method of any one of embodiments 1 to 4, wherein the cancer is chosen from thyroid cancer, non-small cell lung cancer, and other solid tumor cancers.
6. The method of any one of embodiments 1 to 4, wherein the cancer is chosen from medullary thyroid cancer and lung adenocarcinoma.
7. The method of any one of embodiments 1 to 4, wherein the cancer is chosen from papillary thyroid carcinoma (PTC), medullary thyroid cancer (MTC), pheochromocytoma (PCC), pancreatic ductal adenocarcinoma, multiple endocrine neoplasia (MEN2A and MEN2B), metastatic breast cancer, testicular cancer, small cell lung cancer, non-small cell lung cancer (NSCLC), chronic myelomonocytic leukemia (CMML), colorectal cancer, ovarian cancer, inflammatory myofibroblastic tumor, and cancer of the salivary gland.
8. The method of any one of embodiment 1 to 4, wherein the cancer is chosen from esophageal cancer, skin cancer (non-melanoma), endometrial cancer, head and neck cancer, bladder cancer, prostate cancer, hematological cancer, leukemia, soft tissue sarcoma, renal cell carcinoma (RCC), non-Hodgkin lymphoma, hepatobiliary cancer, adrenocortical carcinoma, myelodysplasia (MDS), uterine sarcoma, germ cell tumor, cervical cancer, central nervous system cancer, bone cancer, ampullary carcinoma, gastrointestinal stromal tumor, small bowel cancer, mesothelioma, rectal cancer, paraganglioma, and intrahepatic bile duct cancer.
9. The method of any one of embodiments 1 to 4, wherein the cancer is chosen from adenocarcinoma, spitzoid neoplasm, lung adenocarcinoma, adenosquamous carcinoma, colon cancer, metastatic colon cancer, metastatic papillary thyroid cancer, diffuse sclerosing variant of papillary thyroid cancer, primary myelofibrosis with secondary acute myeloid leukemia, diffuse gastric cancer, thyroid gland carcinoma, and bronchioles lung cell carcinoma.
10. The method of any one of embodiments 1 to 4, wherein the cancer is chosen from hepatobiliary cancer, ampullary carcinoma, small bowel cancer, intrahepatic bile duct cancer, metastatic colon cancer, brain cancer associated with lung cancer, brain metastasis associated with lung cancer, and retropentoneal paraganglioma.
11. The method of any one of embodiments 1 to 4, wherein the cancer is chosen from medullary thyroid cancer (MTC) and non-small cell lung cancer (NSCLC).
12. The method of embodiment 11, wherein the cancer is chosen from sporadic MTC, metastatic RET-altered NSCLC, tyrosine kinase inhibitor (TKI)-refractory KIF5B-RET NSCLC, and KIF5B-RET NSCLC.
13. The method of any one of embodiments 1 to 4, wherein the cancer is chosen from a brain cancer associated with a lung cancer.
14. The method of embodiment 13, wherein the brain cancer is brain metastasis.
15. The method of any one of embodiments 1 to 4, wherein the cancer is RET-altered medullary thyroid cancer (MTC).
16. The method of embodiment 15, wherein the cancer is familial MTC.
17. The method of embodiment 15, wherein the cancer is sporadic MTC.
18. The method of any one of embodiments 1 to 4, wherein the cancer is MTC having a M918T mutation.
19. The method of any one of embodiments 1 to 4, wherein the cancer is MTC having a C634R mutation.
20. The method of any one of embodiments 1 to 3, wherein the cancer is MTC having a V804M mutation.
21. The method of embodiment 1 or 2, wherein the cancer is paraganglioma.
22. The method of embodiment 21, wherein the cancer is retropentoneal paraganglioma.
23. The method of embodiment 21 or 22, wherein the paraganglioma has a R77H mutation.
24. The method of any one of embodiments 1 to 4, wherein the cancer is RET-altered NSCLC.
25. The method of embodiment 24, wherein the cancer is NSCLC having a KIF5B-RET fusion.
26. The method of embodiment 24, wherein the cancer is NSCLC having a CCDCl6-RET fusion.
27. The method of embodiment 24, wherein the cancer is NSCLC having a KIAA1468-RET fusion.
28. The method of embodiment 24, wherein the cancer is NSCLC having a RET fusion identified as FISH positive.
29. The method of any one of embodiments 1 to 4, wherein the cancer is RET-altered PTC.
30. The method of embodiment 29, wherein the cancer is PTC having a CCDCl6-RET fusion.
31. The method of embodiment 29, wherein the cancer is PTC having a NCOA4-RET fusion.
32. The method of embodiment 1 or 2, wherein the cancer is RET-altered intrahepatic bile duct carcinoma.
33. The method of embodiment 32, wherein the cancer is intrahepatic bile duct carcinoma having a NCOA4-RET fusion.
34. The method of any one of embodiments 1 to 33, wherein the subject has not received prior treatment with a multikinase RET inhibitor.
35. The method of any one of embodiments 1 to 33, wherein the subject has received one or more prior treatments with a multikinase RET inhibitor.
36. The method of embodiment 35, wherein the multikinase RET inhibitor is chosen from lenvatinib, vandetanib, cabozantinib, and RXDX-105.
37. The method of any one of embodiments 1 to 36, wherein the subject has not received prior chemotherapy.
38. The method of any one of embodiments 1 to 36, wherein the subject has received prior chemotherapy.
39. The method of embodiment 38, wherein the prior chemotherapy is chosen from carboplatin, pemetrexed, abraxane, cisplatin, bevacizumab, and combinations thereof.
40. The method of any one of embodiments 1 to 39, wherein the subject has not received prior immunotherapy.
41. The method of any one of embodiments 1 to 39, wherein the subject has received prior immunotherapy.
42. The method of embodiment 41, wherein the prior immunotherapy is chosen from ipilimumab, pembrolizumab, nivolumab, MPDL3280A, MEDI4736, and combinations thereof.
43. The method of any one of embodiments 1 to 42, wherein the mTORC1 inhibitor is everolimus or AZD8055.
44. The method of any one of embodiments 1 to 43, wherein the mTORC1 inhibitor is everolimus.
45. The method of any one of embodiments 1 to 43, wherein mTORC1 inhibitor is AZD8055.
46. The method of any one of embodiments 1 to 45, wherein the RET inhibitor has the structure:

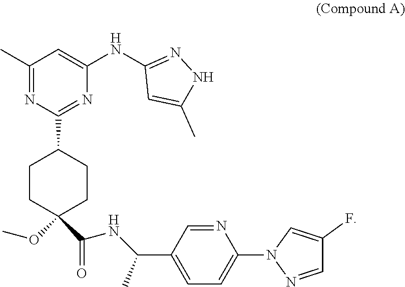 [SMILES: CO[C@]1(C(=O)N[C@@H](C)c2ccc(-n3cc(F)cn3)nc2)CC[C@H](c2nc(C)cc(Nc3cc(C)[nH]n3)n2)CC1]

47. The method of any one of embodiment 1 to 46, wherein:

the subject is a human;

the RET inhibitor is Compound A and the amount administered is between about 5 mg/day and about 1000 mg/day; and

the amount of mTORC1 inhibitor administered is between about 1 mg/day to about 1000 mg/day.

48. The method of embodiment 47, wherein the amount of Compound A administered is 60 mg to 400 mg once daily.
49. The method of embodiment 47 or 48, wherein the amount of Compound A administered is 100 mg to 400 mg once daily.
50. The method of any one of embodiments 47 to 49, wherein the amount of Compound A administered is 300 mg to 400 mg once daily.
51. The method of any one of embodiments 47 to 50, wherein the amount of Compound A administered is 300 mg once daily.
52. The method of any one of embodiments 47 to 50, wherein the amount of Compound A administered is 400 mg once daily.
53. The method of any one of embodiments 47 to 52, wherein the mTORC1 inhibitor is everolimus or AZD8055.
54. The method of any one of embodiments 47 to 53, wherein the mTORC1 inhibitor is everolimus.
55. The method of any one of embodiments 47 to 53, wherein mTORC1 inhibitor is AZD8055.
56. A combination comprising:

a RET inhibitor; and

an mTORC1 inhibitor,

wherein the RET inhibitor and the mTORC1 inhibitor are formulated into:
a single dosage form; or
separate dosage forms which are combined in a kit.
   57. The combination of embodiment 56, wherein the mTORC1 inhibitor is everolimus or AZD8055.
   58. The combination of embodiment 56 or 57, wherein the mTORC1 inhibitor is everolimus.
   59. The combination of embodiment 56 or 57, wherein the mTORC1 inhibitor is AZD8055.
   60. The combination of any one of embodiments 56 to 59, wherein the RET inhibitor has the structure:

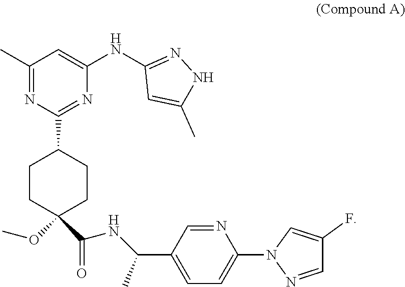 [SMILES: CO[C@]1(C(=O)N[C@@H](C)c2ccc(-n3cc(F)cn3)nc2)CC[C@H](c2nc(C)cc(Nc3cc(C)[nH]n3)n2)CC1]

61. The combination of any one of embodiments 56 to 60, wherein:

the RET inhibitor and the mTORC1 inhibitor are formulated into separate dosage forms which are combined in a kit; and

the kit further comprises instructions for co-administering the RET inhibitor and the mTORC1 inhibitor for the treatment of a cancer mediated by aberrant RET activity.

62. A method of treating a cancer mediated by aberrant RET activity in a subject in need thereof, the method comprising co-administering to the subject a RET inhibitor and an mTORC1 inhibitor, wherein administration of the RET inhibitor is associated with a sustained down-regulation of at least one effect marker in the subject.
63. The method of embodiment 62, wherein the RET inhibitor is Compound A.
64. The method of embodiment 62 or 63, wherein the mTORC1 inhibitor is everolimus or AZD8055.
65. The method of any one of embodiments 62 to 64, wherein the mTORC1 inhibitor is everolimus.
66. The method of any one of embodiments 62 to 64, wherein the mTORC1 inhibitor is AZD8055.
67. The method of any one of embodiments 62 to 66, wherein the effect marker is chosen from DUSP6 mRNA expression, SPRY4 mRNA expression, carcinoembryonic antigen level, and calcitonin level.
68. The method of any one of embodiments 62 to 66, wherein the effect marker is KIF5B ctDNA level or TP53 ctDNA level.
69. The method of any one of embodiments 62 to 68, wherein the amount administered to the subject produces a greater than 95% down-regulation of at least one effect marker.
70. The method of any one of embodiments 62 to 68, wherein the amount administered to the subject produces a greater than 94%, greater than 93%, greater than 92%, greater than 91%, greater than 90%, greater than 89%, greater than 88%, greater than 87%, greater than 86% greater than 85%, greater than 80%, greater than 75%, greater than 70%, greater than 65%, greater than 60%, greater than 55%, or greater than 50% down-regulation in at least one effect marker.
71. The method of any one of embodiments 62 to 68, wherein the amount administered to the subject produces a greater than 89%, greater than 88%, greater than 87%, greater than 86%, greater than 85%, greater than 80%, greater than 75%, or greater than 70% down-regulation in at least one effect marker.
72. The method of any one of embodiments 62 to 71, wherein at least two effect markers are down-regulated.

In some embodiments, each of the RET inhibitor and the mTORC1 inhibitor are formulated into a pharmaceutical composition. The two inhibitors may be formulated into separate compositions or combined in a single composition. If formulated into separate compositions, those compositions may be formulated for either the same route of delivery or for different routes of delivery. In some embodiments, each of the RET inhibitor and mTORC1 inhibitor are formulated for the same route of delivery in which that inhibitor would be used in a monotherapy. In some embodiments, the RET inhibitor is formulated for oral delivery. In some embodiments, the mTORC1 inhibitor is formulated for oral delivery. In some embodiments, both the RET inhibitor and the mTORC1 inhibitor are formulated for oral delivery.

Pharmaceutical compositions of the disclosure comprise one or more compounds of the disclosure and one or more physiologically or pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable carrier,” as used herein, refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Compositions of the disclosure may be administered orally, parenterally, by inhalation spray, topically, rectally, nasally, buccally, vaginally, or via an implanted reservoir. The term “parenteral,” as used herein, includes subcutaneous, intravenous, intramuscular, intra-articular, intra-synovial, intrasternal, intrathecal, intrahepatic, intralesional, and intracranial injection, or infusion techniques. In some embodiments, the compositions of the disclosure are administered orally, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tween, Spans, and other emulsifying agents or bioavailability enhancers which are commonly used in the manufacture of pharmaceutically acceptable solid, liquid, or other dosage forms may also be used for the purposes of formulation.

The pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents may also be added.

Alternatively, the pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax, and polyethylene glycols.

The pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be affected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax, and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of the compounds of the present disclosure that may be combined with the carrier materials to produce a composition in a single dosage form will vary depending upon the host treated, the particular mode of administration.

In some embodiments, the dosage of each of the RET inhibitor and the mTORC1 inhibitor are equal to the dose of each when used in a monotherapy.

As demonstrated herein, the combination of a RET inhibitor and an mTORC1 inhibitor in the treatment of cancers characterized by aberrant RET activity show unexpected synergy. Because of that synergy, it is possible to use dosages of RET inhibitor and/or mTORC1 inhibitor that are less than those used in a monotherapy. Accordingly, in some embodiments, the dosage of the RET inhibitor used in the methods of this disclosure is less than 95%, less than 90%, less than 85%, less than 80%, less than 75%, or less than 70% of the dose used when such RET inhibitor is used in a monotherapy. Additionally, in some embodiments, the dosage of the mTORC1 inhibitor used in the methods of this disclosure is less than 95%, less than 90%, less than 85%, less than 80%, less than 75%, or less than 70% of the dose used when such mTORC1 inhibitor is used in a monotherapy. In some embodiments, both the dosage of the RET inhibitor and the mTORC1 inhibitor used in the methods of this disclosure is less than 95%, less than 90%, less than 85%, less than 80%, less than 75%, or less than 70% of the dose used when each of such inhibitors is used in a monotherapy.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present disclosure in the composition will also depend upon the particular compound in the composition.

In some embodiments, the dosage of each the compounds of the combination used in the methods described herein is between about 5 mg to about 1000 mg. In some embodiments, when the RET inhibitor is Compound A, the dosage used in the methods of this disclosure is between about 5 mg to about 1000 mg.

In some embodiments, the amount of Compound A administered is 60 mg to 400 mg.

In some embodiments, the amount of Compound A administered is 100 mg to 400 mg.

In some embodiments, the amount of Compound A administered is 300 mg to 400 mg.

In some embodiments, the amount of Compound A administered is 300 mg.

In some embodiments, the amount of Compound A administered is 400 mg.

In some embodiments, Compound A is administered once daily.

In some embodiments, the amount of Compound A administered is 60 mg to 400 mg once daily.

In some embodiments, the amount of Compound A administered is 100 mg to 400 mg once daily.

In some embodiments, the amount of Compound A administered is 300 mg to 400 mg once daily.

In some embodiments, the amount of Compound A administered is 300 mg once daily.

In some embodiments, the amount of Compound A administered is 400 mg once daily.

In some embodiments, the disclosure features a kit (e.g., a pharmaceutical pack) comprising: a RET inhibitor; and an mTORC1 inhibitor, wherein the RET inhibitor and the mTORC1 inhibitor are each formulated into separate dosage forms. The kits are useful for treating a cancer characterized by aberrant RET activity, e.g., the cancers described herein. The kits provided may comprise each of the mTORC1 inhibitor and RET inhibitor in a separate container (e.g., a vial, ampule, bottle, syringe, and/or dispenser package, or other suitable container). In some embodiments, the provided kits may optionally further include additional containers comprising a pharmaceutical excipient for dilution or suspension of one or both of the RET inhibitor and the mTORC1 inhibitor. In some embodiments, the mTORC1 inhibitor and RET inhibitor in separate containers are combined (optionally with a third container comprising a pharmaceutical excipient for dilution or suspension) to form one unit dosage form prior to administration.

The kit may further include written instructions for administration of the two inhibitors (e.g., how to combine the two inhibitors into a single dosage form, the types of cancer for which the kit is useful, the frequency of administration of each of the mTORC1 and RET inhibitors as separate dosage forms, and other information relevant to the co-administration of the two inhibitors.

### EXAMPLES

The following examples are intended to be illustrative and are not meant in any way to be limiting.

### Example 1. In Vitro Cell Proliferation Assays

The cell lines used in the studies described below were purchased from ATCC (TT cells, RET C634W medullary thyroid cancer cell line), Riken (LC2/ad cells, CCDCl6-RET lung adenocarcinoma cell line), or Sigma (FTC-133, follicular thyroid carcinoma), or acquired from the University of Colorado (MZ-CRC-1, RET M918T, medullary thyroid cancer cells).

Cells were plated in duplicate in 96-well opaque, clear-bottom plates (Falcon) using a Multidrop (Thermo Fisher) and treated with compound combinations as described below on the following day. Triplicate wells for DMSO and the kinase inhibitor staurosporine were included on each plate to determine relative inhibition of cell proliferation. Cell lines were incubated for 3 days (FTC-133) or 5 days (TT, MZ-CRC-1, LC2/ad) and relative proliferation was measured using the CellTiter-Glo (CTG) assay (Promega). To calculate percent inhibition of cell proliferation, the following formula was used:

1−([(average combination well signal)−(average staurosporine signal)]/[(average DMSO signal)−(average staurosporine signal)])

### Example 2. Combination Studies

Potential synergistic interactions between compounds were assessed relative to the Loewe additivity model using CHALICE™ software (Horizon CombinatoRx Inc., Cambridge, Mass.), via a synergy score calculated from the difference between the observed values and those predicted from the Loewe additivity model across a range of concentrations of each compound. For these experiments, a minimum of a 5 point dilution series of Compound A (dose range 1000 nM-0.39 nM) was combined with a 7 point dilution series of AZD-8055 with a dose range 1000 nM-1.4 nM to create drug combination matrices that spanned 48-64 distinct combinations. Percent inhibition of cell proliferation values were calculated as described above and entered into the CHALICE software to generate a synergy score and an isobologram to visualize any excess inhibition or potency shifts obtained with compounds tested in combination. Synergistic growth inhibition occurred when the combined effect of two compounds was greater than what was predicted based on the Loewe additivity model for the compound combination. This method of assessing synergistic growth inhibition is explained in detail in Lehar et al. (Nat. Biotechnology, 2009) and the CHALICE software technical guide.

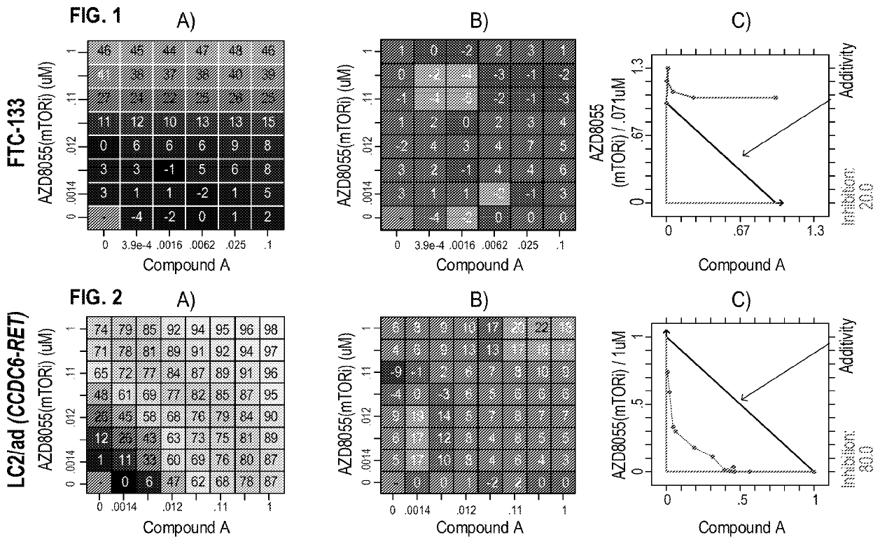

In FIGS. 1-4, panel A, the matrices of inhibition values measured with the CTG assay is shown for each cell line in the presence of a single compound and across a range of combination concentrations. In FIGS. 1-4, panel B, the Loewe Excess inhibition matrices and in FIGS. 1-4, panel C, the isobolograms indicate the excess inhibition observed over the inhibition predicted by the Loewe additivity model. In the isobolograms, the straight line connecting the abscissa and the ordinate values represents growth inhibitions that were strictly additive for the combination of the two compounds. Plots that fall below the straight line represented synergistic growth inhibitions.

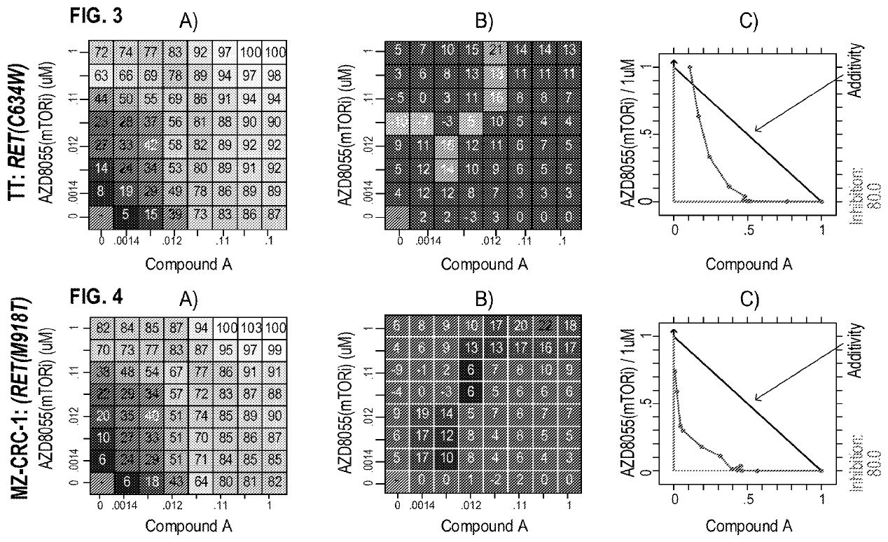

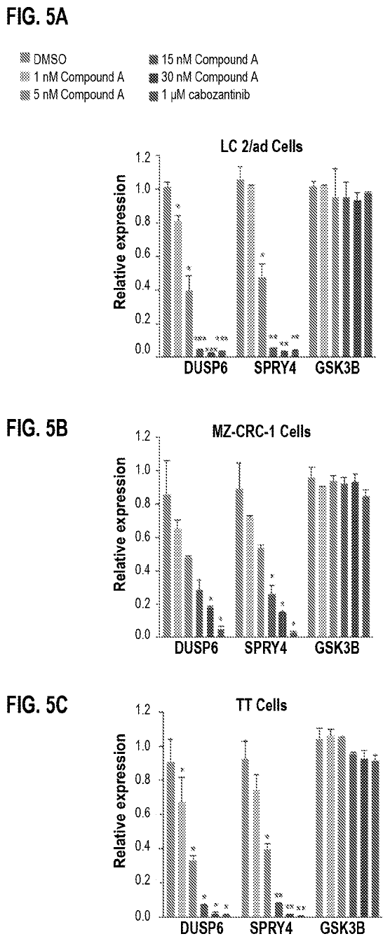

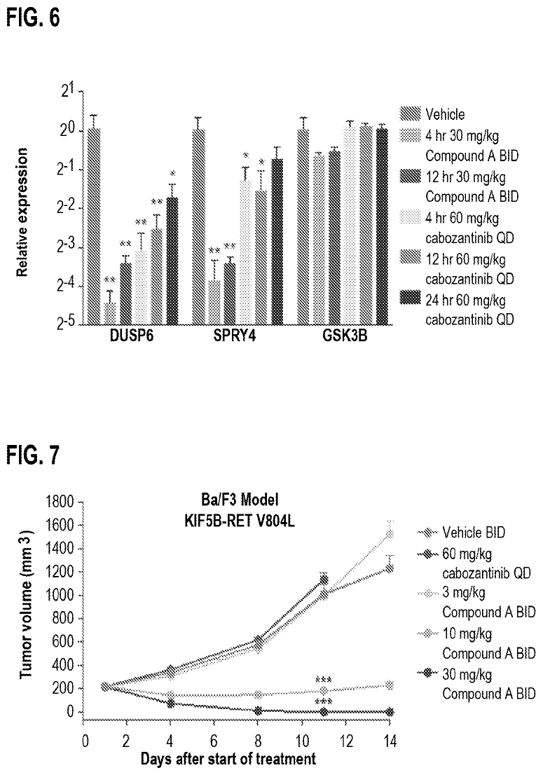

As can be observed in FIGS. 2-4 synergistic interaction is observed for the combination of Compound A and AZD8055 in RET-driven cell lines, including the medullary thyroid cancer cell lines TT (FIG. 3) and MZ-CRC-1 (FIG. 4), and the lung adenocarcinoma cell line LC2/ad (FIG. 2). Synergistic interactions were not observed in the FTC-133 cell line (FIG. 1), a cell line that does not have an oncogenic RET mutation and is not sensitive to RET inhibition.

A summary of the overall synergy score for the combination of Compound A and AZD8055 is shown in Table 4, below. Synergistic interaction occurs for synergy scores greater than 1.

TABLE 4
Summary of Synergy Scores of Compound
A and AZD8055 in Various Cell Lines

| Cell Line | RET Alteration | Synergy Score |
|---|---|---|
| FTC-133 | None | 0.107 |
| LC2/ad | CCDC6-RET | 5.27 |
| TT | RET(C634W) | 3.86 |
| MZ-CRC-1 | RET(M918T) | 4.02 |

Additionally, the following examples further support the use of Compound A in the treatment of RET-altered cancers.

### Example 3. DUSP6 and SPRY4 Expression Analysis

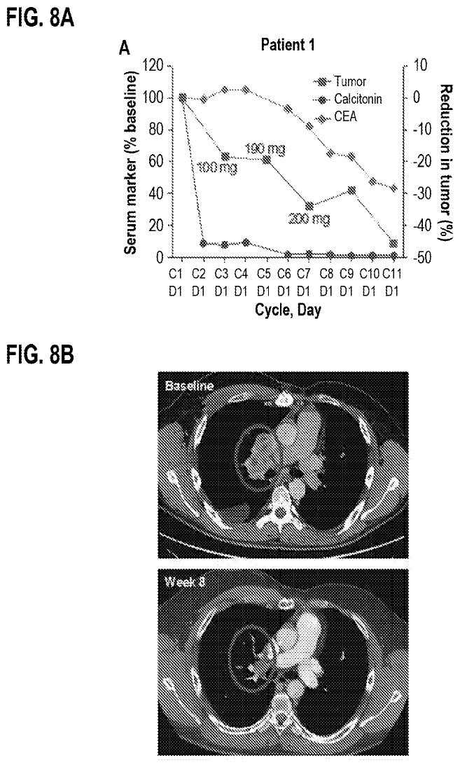

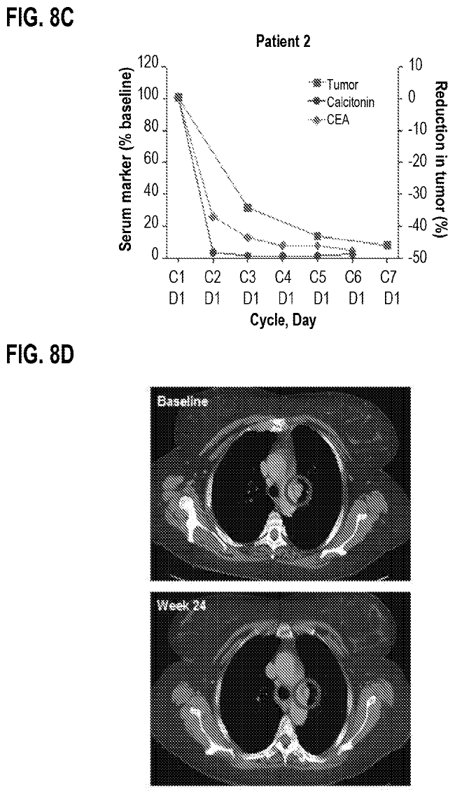

Cells were treated with the indicated compounds for 7 hours before lysis with Buffer RLT (QIAGEN, Hilden, Germany) containing 1% β-mercaptoethanol. Total RNA was isolated using the Rneasy Plus Mini kit (QIAGEN, Hilden, Germany) according to the manufacturer's instructions. First-strand cDNA was synthesized using the SuperScript VILO Master Mix (Thermo Fisher Scientific, Waltham, Mass.) according to the manufacturer's instructions. Real-time qPCR was run on ViiA 7 Real Time PCR System (Thermo Fisher Scientific). For qRT-PCR, the expression of the reference gene glucuronidase beta (GUSB) was used to normalize expression of the target genes DUSP6, SPRY4, and glycogen synthase kinase 3 beta (GSK3B). Replicate qRT-PCR reactions were analyzed for each sample, and QuantStudio Real-Time PCR software (Life Technologies, Carlsbad, Calif.) normalized the average expression of DUSP6, SPRY4, or GSK3B to the average expression of the reference gene GUSB in each sample. FIGS. 5A-5C show relative transcript expression of RET pathway targets DUSP6 and SPRY4 and AKT-pathway target GSK3B 7 hours after treatment of (FIG. 5A) L2C/ad, (FIG. 5B) MZ-CRC-1 cells, or (FIG. 5C) TT MTC cells with Compound A or cabozantinib. FIG. 6 shows relative transcript expression of DUSP6, SPRY4 and GSK3B from KIF5B-RET NSCLC PDX. Tumors collected at the indicated times (hours) after administration of last dose. Data are the mean+SD. *P<0.05, **P<0.01, ***P<0.001, 2-sided Student's t-test. SD, standard deviation.

### Example 4. Generation of KIF5B-RET Ba/F3 Cells and ENU Mutagenesis Assays

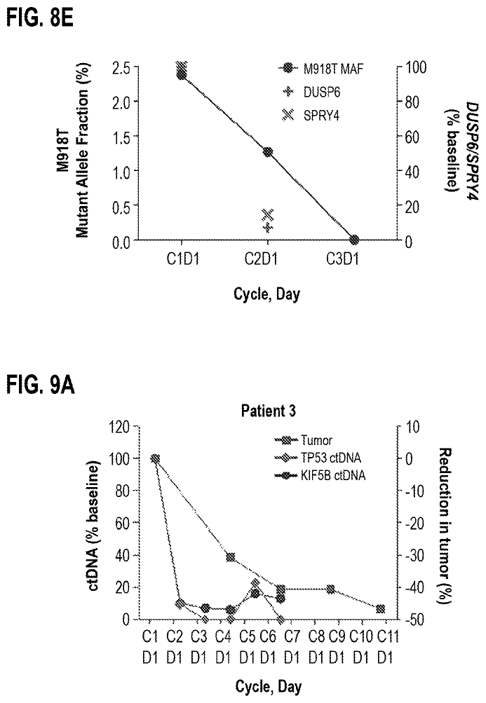

The DNA encoding the amino acid sequence of human KIF5B-RET variant 1 was placed in a lentivirus vector under a doxycycline-inducible promoter to maximize expression with a carboxyl-terminal FLAG epitope to facilitate immunodetection of the fusion by anti-FLAG antibodies. Lentiviral-mediated gene transduction was used to express KIF5B-RET in Ba/F3 cells, KIF5B-RET dependent cells were selected by IL-3 withdrawal and confirmed to express the KIF5B-RET fusion protein by immunoblot analysis. To generate Ba/F3 cells carrying V804 substitutions, WT KIF5B-RET Ba/F3 cells were mutagenized overnight with ENU and plated in 96-well plates for a period of 2 weeks in the presence of 6 concentrations of MKIs (ponatinib, regorafenib, cabozantinib, or vandetanib). The concentrations chosen ranged from 2×-64× the proliferation IC₅₀ for each compound: 125 nM to 4 μmol/L cabozantinib, 20 to 640 nM ponatinib, and 250 nM to 8 μmol/L vandetanib. Genomic DNA was isolated from resistant clones, and Sanger sequencing was used to identify those that harbored substitutions. FIG. 7 shows antitumor activity of Compound A compared with cabozantinib in KIF5B-RET V804L Ba/F3 allografts.

### Example 5. Phase I Study

A phase 1, first-in-human study (NCT03037385) to define the maximum tolerated dose, safety profile, pharmacokinetics, and preliminary anti-tumor activity of Compound A in advanced, RET-altered NSCLC, MTC and other solid tumors was initiated. Prior to study entry, written informed consent was obtained from all patients for treatment with Compound A and collection of blood and tumor samples for exploratory biomarker analyses to characterize potential predictive biomarkers of safety and efficacy. Adult patients (218 years of age) must have had advanced, unresectable solid tumors, with an Eastern Cooperative Oncology Group performance status of 0 to 2, and adequate bone marrow, hepatic, renal, and cardiac function. Compound A was administered orally, once daily, on a 4-week cycle using a Bayesian Optimal Interval Design. At dose levels 2120 mg, documented RET-alteration was additionally required for study entry. Adverse events were graded per Common Terminology Criteria for Adverse Events (CTCAE). Radiographic response by computed tomography was evaluated RECIST version 1.1 (European Journal of Cancer 45: 228-247 (2009)). Levels of ctDNA in plasma were assessed using the PlasmaSELECT™-R64 NGS panel (Personal Genome Diagnostics, Baltimore, Md.). Serum calcitonin levels in MTC patients were measured by ELISA (Medpace, Cincinnati, Ohio). Tumor DUSP6/SPRY4 levels were analyzed by qRT-PCR (Molecular MD, Portland, Oreg.).

### Case Studies

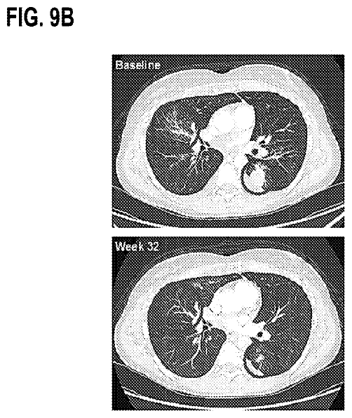

Patient 1 was a 27-year-old patient with sporadic MTC harboring multiple RET mutations (L629P, D631_R635DELINSG, and V637R). The patient was tyrosine kinase inhibitor naïve prior to the start of Compound A treatment with highly invasive disease that required emergent tracheostomy and extensive surgery, including total thyroidectomy, central neck dissection, bilateral levels 1 through 4 neck dissection, total thymectomy, and median sternotomy. The postoperative course was complicated by chylothorax. Multidisciplinary medical consensus was against radiotherapy to the neck, and restaging scans showed left paratracheal disease with tracheal and esophageal invasion as well as metastatic disease to the lungs and liver. The two FDA approved multi-kinase drugs for MTC (vandetanib and cabozantinib) were not considered appropriate for this patient given the associated risk of VEGFR-related toxicities that can include impaired wound healing, and increase the risk of fistula formation and hemorrhage (CAPRELSA (vandetanib) [package insert]. Cambridge, Mass.: Sanofi Genzyme; 2016; COMETRIQ (cabozantinib) [package insert]. South San Francisco, Calif.: Exelixix, Inc.; 2018). Therefore, the patient was enrolled on the Compound A clinical trial and began treatment at the second dose level (60 mg, QD). Remarkably, after 28 days of Compound A therapy, there was a >90% reduction in the serum tumor marker calcitonin (FIG. 8A). After 8 weeks, target lesions were reduced by 19%. After successive dose escalations of Compound A to 200 mg QD, the patient achieved partial response with >30% tumor reduction per Response Evaluation Criteria in Solid Tumors (RECIST) version 1.1 (FIG. 8B). This patient subsequently escalated to 300 mg QD Compound A and achieved a confirmed partial response (47% maximal reduction) at 10 months. Overall, carcinoembryonic antigen (CEA) levels decreased by 57% over this period. Improved health status with Compound A treatment allowed for removal of the patient's tracheostomy tube and a return to baseline body weight after several kilograms of weight loss prior to treatment. Compound A has been well tolerated throughout 11 months of continuous treatment with the only drug-related adverse event being transient grade 1 decrease in white blood cells, which resolved without drug interruption or dose modification. As of Apr. 13, 2018, the patient remained on therapy.

Patient A was a 56-year-old with sporadic RET M918T-mutant MTC, who had responded and then progressed on vandetanib, initiated therapy with Compound A, 300 mg QD. Early signals of clinical activity emerged within the first few weeks of Compound A treatment: serum calcitonin decreased >90% and CEA decreased by 75% after 28 days (FIG. 8C). RET M918T circulating tumor DNA (ctDNA) decreased by 47% after 28 days and was not detectable after 56 days. Paired tumor biopsies collected pretreatment and 28 days post-treatment demonstrated a 93% reduction in DUSP6 and an 86% reduction in SPRY4 mRNA expression, confirming RET-pathway inhibition within the tumor (FIG. 8E). Importantly, these indications of activity were confirmed by radiographic response (−35%) per RECIST 1.1 after 8 weeks (FIG. 8D). The patient tolerated Compound A treatment well without dose interruption; drug-related adverse events were grade 1 nausea and hyperphosphatemia. The patient continued on therapy at 8 months with a confirmed partial response (maximum 47% reduction) as of Apr. 13, 2018.

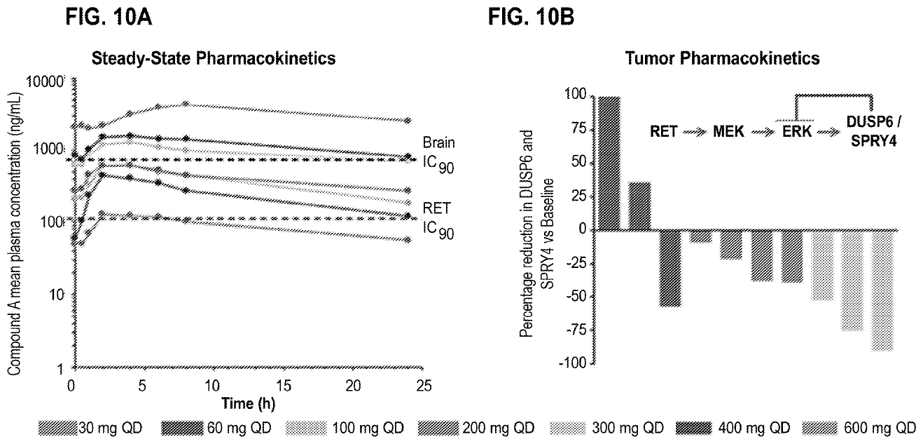

Patient 3 was a 37-year-old patient with metastatic RET-altered NSCLC, who had progressed on cisplatin, pemetrexed, and bevacizumab, had tumor tissue test positive for a RET fusion via FISH analysis. The patient initiated treatment with 200 mg QD Compound A, and ctDNA analysis at baseline revealed a canonical KIF5B-RET fusion and co-occurring TP53 mutation. Tumor reduction (−25%) was noted at first radiographic assessment after 8 weeks of treatment and correlated with a concomitant decline in KIF5B-RET and TP53 ctDNA levels (FIG. 9A). The patient achieved a partial response on the second radiographic assessment after 16 weeks (FIG. 9B) and continued on treatment through 10 months with a confirmed partial response as of Apr. 13, 2018. As observed with the MTC patients described above, Compound A has been well tolerated, with all drug-related adverse events being grade 1 and including constipation (resolved), dry skin, rash, and leukopenia.

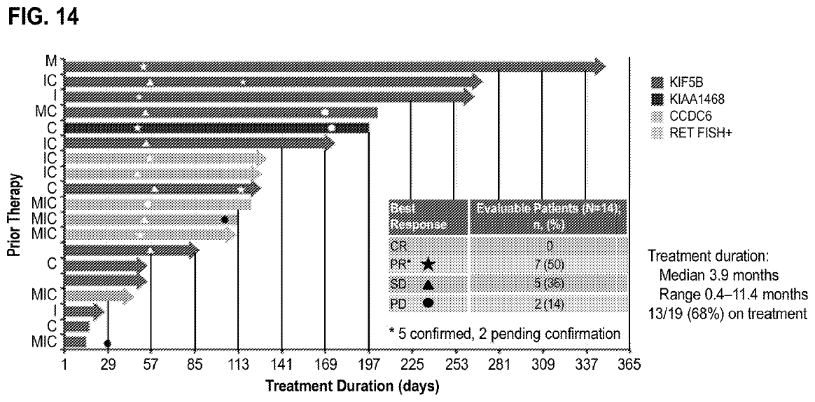

Patient 4 was a 69-year-old patient with NSCLC, who had prior lung resection nephrectomy, and pleural drainage. The patient initiated treatment with 400 mg QD Compound A. Tumor reduction was noted against KIF5B-RET NSCLC brain metastases (FIG. 13). Specifically, evidence of intracranial anti-tumor activity was observed in the patient. At baseline, the patient had an approximately 6 mm metastatic lesion in the brain, which appeared to resolve after 8 weeks on treatment. At the time of the 8-week assessment, the patient was determined to have stable disease.

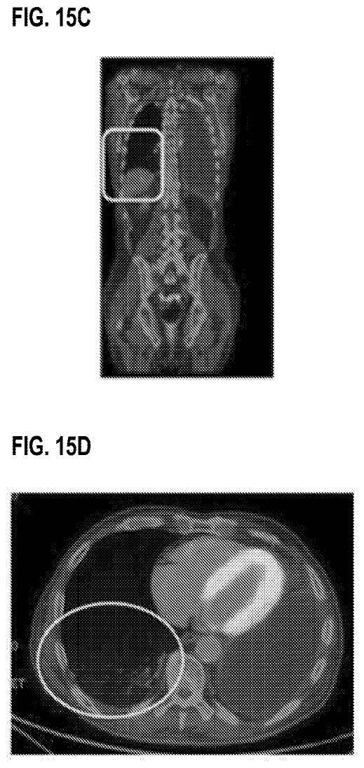

Patient 5 was a 74-year-old former smoker with locally advanced KIF5B-RET NSCLC. The patient's CT scans are shown in FIGS. 15A-D. The patient had received concurrent chemoradiation with cisplatin and pemetrexed, was then treated with carboplatin and nab-paclitaxel and eventually progressed. Next generation sequencing of the tumor tissue, along with FISH, revealed a KIF5B-RET fusion, and the patient was enrolled on a clinical trial testing a combination regimen of vandetanib and everolimus (NCT01582191). The patient achieved a partial response, but restaging scans performed after 11 cycles showed progressive disease, which was associated with clinical symptoms of increasing dyspnea and worsening performance status. The patient was then enrolled on the phase 1 trial of Compound A. After 16 weeks of treatment with Compound A (300 mg QD), the patient had a partial response with 34% reduction of tumor volume (FIGS. 15C and 15D) and improvement of dyspnea and performance status. Compound A has been well tolerated throughout treatment, and the patient had not experienced drug-related adverse events as of Apr. 13, 2018.

Measuring ctDNA Levels

Levels of one example effect marker, ctDNA in plasma (e.g., KIF5B or TP53 ctDNA), may be assessed using the PlasmaSELECT™-R64 NGS panel (Personal Genome Diagnostics, Baltimore, Md.). PlasmaSELECT™64 analyzes circulating tumor DNA for genetic alterations in cancer. Specifically, PlasmaSELECT™64 evaluates a targeted panel of 64 well-characterized cancer genes. Cell-free DNA is extracted from plasma and prepared using proprietary methods that accommodate low abundance sample DNA. Samples are then processed using a proprietary capture process and high coverage next-generation sequencing.

Steady State Plasma Concentration, RET IC₉₀ and Brain IC₉₀ (Predicted)

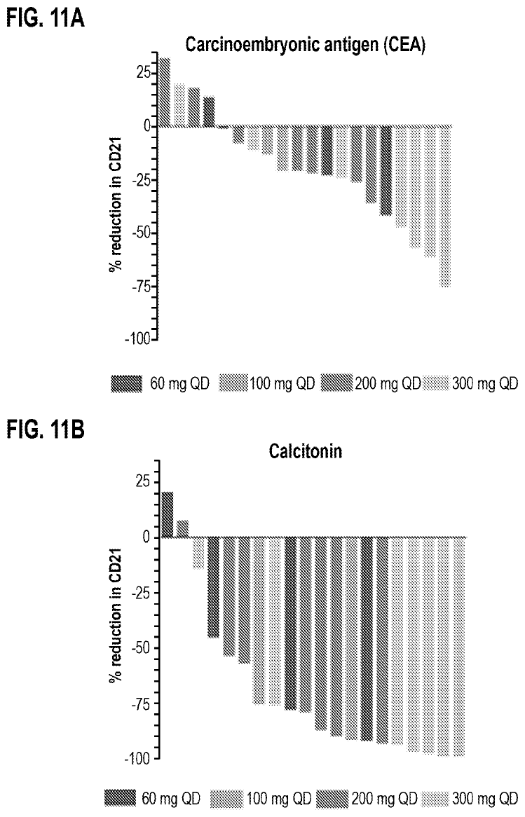

Blood samples were collected at pre-determined time points from patients dosed with 30 to 600 mg Compound A orally once daily. Plasma samples were analyzed for Compound A using a validated liquid chromatography-tandem mass spectrometry (LC-MS/MS) method. The plasma Compound A concentration-time data were graphed using Phoenix WinNonlin© (Version 6.4, Certara L.P.) or Graphpad Prism (Version 7.02). FIG. 10A shows the plasma concentration-time profile of Compound A at steady state. The RET IC₉₀ and brain IC₉₀ (predicted) are based on projections and extrapolations based on PK and PD data in animals.

All publications and patent applications mentioned herein are hereby incorporated by reference in their entirety as if each individual publication or patent was specifically and individually indicated to be incorporated by reference.

Those skilled in the art will recognize or be able to ascertain using no more than routine experimentation many equivalents to the specific embodiments of the disclosure described herein. Such equivalents are intended to be encompassed by the following claims.

1. A method of treating a cancer mediated by aberrant RET activity in a subject in need thereof, the method comprising co-administering to the subject a RET inhibitor and an mTORC1 inhibitor, wherein the RET inhibitor has the structure:

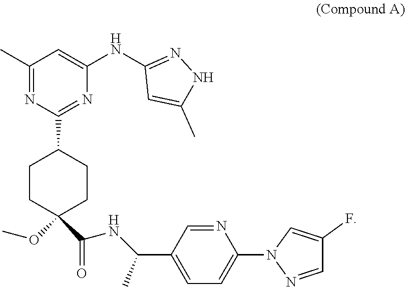 [SMILES: CO[C@]1(C(=O)N[C@@H](C)c2ccc(-n3cc(F)cn3)nc2)CC[C@H](c2nc(C)cc(Nc3cc(C)[nH]n3)n2)CC1]

2. (canceled)

3. The method of claim 1, wherein the aberrant RET activity is attributed to:
a RET mutation chosen from C634W, V804L, V804M, V804E, Y806C, Y806S, Y806H, Y806N, G810R, G810S, L865V, L870F, S891A and M918T; or
a RET fusion chosen from KIF5B-RET and CCDC6-RET.

4. The method of claim 1, wherein the aberrant RET activity is attributed to:
a RET mutation chosen from C634W and M918T; or
a CCDCl6-RET fusion.

5. The method of claim 1, wherein the cancer is chosen from thyroid cancer, non-small cell lung cancer, and other solid tumor cancers.

6. (canceled)

7. The method of claim 1, wherein the cancer is chosen from papillary thyroid carcinoma (PTC), medullary thyroid cancer (MTC), pheochromocytoma (PCC), pancreatic ductal adenocarcinoma, multiple endocrine neoplasia (MEN2A and MEN2B), metastatic breast cancer, testicular cancer, small cell lung cancer, non-small cell lung cancer (NSCLC), chronic myelomonocytic leukemia (CMML), colorectal cancer, ovarian cancer, inflammatory myofibroblastic tumor, and cancer of the salivary gland.

8. The method of claim 1, wherein the cancer is chosen from esophageal cancer, skin cancer (non-melanoma), endometrial cancer, head and neck cancer, bladder cancer, prostate cancer, hematological cancer, leukemia, soft tissue sarcoma, renal cell carcinoma (RCC), non-Hodgkin lymphoma, hepatobiliary cancer, adrenocortical carcinoma, myelodysplasia (MDS), uterine sarcoma, germ cell tumor, cervical cancer, central nervous system cancer, bone cancer, ampullary carcinoma, gastrointestinal stromal tumor, small bowel cancer, mesothelioma, rectal cancer, paraganglioma, and intrahepatic bile duct cancer.

9. The method of claim 1, wherein the cancer is chosen from adenocarcinoma, spitzoid neoplasm, lung adenocarcinoma, adenosquamous carcinoma, colon cancer, metastatic colon cancer, metastatic papillary thyroid cancer, diffuse sclerosing variant of papillary thyroid cancer, primary myelofibrosis with secondary acute myeloid leukemia, diffuse gastric cancer, thyroid gland carcinoma, and bronchioles lung cell carcinoma.

10. The method of claim 1, wherein the cancer is chosen from hepatobiliary cancer, ampullary carcinoma, small bowel cancer, intrahepatic bile duct cancer, metastatic colon cancer, brain cancer associated with lung cancer, brain metastasis associated with lung cancer, and retropentoneal paraganglioma.

11.-42. (canceled)

43. The method of claim 1, wherein the mTORC1 inhibitor is everolimus or AZD8055.

44. The method of claim 1, wherein the mTORC1 inhibitor is everolimus.

45. The method of claim 1, wherein mTORC1 inhibitor is AZD8055.

46. (canceled)

47. The method of claim 1, wherein:
the subject is a human;
the amount of compound A administered is between about 5 mg/day and about 1000 mg/day; and
the amount of mTORC1 inhibitor administered is between about 1 mg/day to about 1000 mg/day.

48. The method of claim 47, wherein the amount of Compound A administered is 60 mg to 400 mg once daily.

49. The method of claim 47, wherein the amount of Compound A administered is 100 mg to 400 mg once daily.

50. The method of claim 47, wherein the amount of Compound A administered is 300 mg to 400 mg once daily.

51. The method of claim 47, wherein the amount of Compound A administered is 300 mg once daily.

52. The method of claim 47, wherein the amount of Compound A administered is 400 mg once daily.

53. The method of claim 47, wherein the mTORC1 inhibitor is everolimus or AZD8055.

54. The method of claim 47, wherein the mTORC1 inhibitor is everolimus.

55. The method of claim 47, wherein mTORC1 inhibitor is AZD8055.

56. A combination comprising:
a RET inhibitor; and
an mTORC1 inhibitor,
wherein the RET inhibitor and the mTORC1 inhibitor are formulated into:
a single dosage form; or
separate dosage forms which are combined in a kit,
wherein the RET inhibitor is Compound A.

57. The combination of claim 56, wherein the mTORC1 inhibitor is everolimus or AZD8055.

58. The combination of claim 56, wherein the mTORC1 inhibitor is everolimus.

59. The combination of claim 56, wherein the mTORC1 inhibitor is AZD8055.

60. (canceled)

61. The combination of claim 56, wherein:
the RET inhibitor and the mTORC1 inhibitor are formulated into separate dosage forms which are combined in a kit; and
the kit further comprises instructions for co-administering the RET inhibitor and the mTORC1 inhibitor for the treatment of a cancer mediated by aberrant RET activity.

62. A method of treating a cancer mediated by aberrant RET activity in a subject in need thereof, the method comprising co-administering to the subject a RET inhibitor and an mTORC1 inhibitor, wherein administration of the RET inhibitor is associated with a sustained down-regulation of at least one effect marker in the subject, wherein the RET inhibitor is Compound A.

63. (canceled)

64. The method of claim 62, wherein the mTORC1 inhibitor is everolimus or AZD8055.

65. The method of claim 62, wherein the mTORC1 inhibitor is everolimus.

66. The method of claim 62, wherein the mTORC1 inhibitor is AZD8055.

67. The method of claim 62, wherein the effect marker is chosen from DUSP6 mRNA expression, SPRY4 mRNA expression, carcinoembryonic antigen level, and calcitonin level.

68. The method of claim 62, wherein the effect marker is KIF5B ctDNA level or TP53 ctDNA level.

69. The method of claim 62, wherein the amount administered to the subject produces a greater than 95% down-regulation of at least one effect marker.

70. The method of claim 62, wherein the amount administered to the subject produces a greater than 94%, greater than 93%, greater than 92%, greater than 91%, greater than 90%, greater than 89%, greater than 88%, greater than 87%, greater than 86% greater than 85%, greater than 80%, greater than 75%, greater than 70%, greater than 65%, greater than 60%, greater than 55%, or greater than 50% down-regulation in at least one effect marker.

71. The method of claim 62, wherein the amount administered to the subject produces a greater than 89%, greater than 88%, greater than 87%, greater than 86%, greater than 85%, greater than 80%, greater than 75%, or greater than 70% down-regulation in at least one effect marker.

72. The method of claim 62, wherein at least two effect markers are down-regulated.
